(19) United States
(12) Patent Application Publication
(10) Pub. No.: US 2011/0003689 A1
(43) Pub. Date: Jan. 6, 2011

(54) USE OF POLYMERIC GUANIDINE DERIVATIVES FOR THE CONTROL OF UNWANTED MICROORGANISMS IN CROP PROTECTION

(75) Inventors: Hans-Juergen Rosslenbroich, Monheim (DE); Arnd Voerste, Koeln (DE); Martin Schoepfer, Wien (AT); Hilmar Wolf, Langenfeld (DE); Ulrike Wachendorff-Neumann, Neuwied (DE); Peter Dahmen, Neuss (DE); Dirk Ebbinghaus, Wuppertal (DE); Oskar Schmidt, Wien (AT)

Correspondence Address: WORKMAN NYDEGGER;1000 Eagle Gate Tower, 60 East South Temple, Salt Lake City, UT, 84111, US

(73) Assignee: AKA TECHNOLOGY GMBH, Wien (AT)

(21) Appl. No.: 12/809,940

(22) PCT Filed: Dec. 6, 2008
(86) PCT No.: PCT/EP08/10370

§ 371 Date: Sep. 10, 2010

(30) Foreign Application Priority Data
Dec. 19, 2007 (EP) 07150147.2

(51) Int. Cl. A01C 1/06; A01P 3/00; A01N 47/44; A61K 31/428; A01N 43/40; A61K 31/24; A61K 31/185; A01N 55/02; A01N 37/34; A01N 43/84; A01N 43/50; A01N 57/12; A01N 59/26; A01N 43/653; A01N 43/54

(52) U.S. Cl. 504/100; 514/634; 514/367; 514/357; 514/479; 514/538; 514/578; 514/494; 514/528; 514/237.5; 514/390; 514/352; 514/142; 424/605; 514/383; 514/275

(57) ABSTRACT

The present invention relates to the use of polymeric guanidine derivatives for the curative and/or preventive treatment of unwanted microorganisms such as bacteria and phytopathogenic fungi in crop protection including the treatment of seed. The invention furthermore relates to novel active compound combinations comprising at least one polymeric guanidine derivative and at least one further fungicidally active compound, their use for the curative or preventive treatment of unwanted microorganisms in crop protection including the treatment of seed and not least the treated seed itself.

The present invention relates to the use of polymeric guanidine derivatives for the curative and/or preventive treatment of unwanted microorganisms such as bacteria and phytopathogenic fungi in crop protection including the treatment of seed. The invention furthermore relates to novel active compound combinations comprising at least one polymeric guanidine derivative and at least one further fungicidally active compound, their use for the curative or preventive treatment of unwanted micro-organisms in crop protection including the treatment of seed and not least the treated seed itself.

Biocidal polymers based on guanidinium hydrochloride, in particular their action against Escherichia coli bacteria, are already known (cf. WO 01/85676). Furthermore, it is already known that such guanidine derivatives can be used as fungicidal agents (cf. WO 2006/047800). Of particular importance are the polymers Akacid®, i.e. poly[2-(2-ethoxy)ethoxyethylguanidinium chloride], and Akacid Plus®, which was originally described as a 3:1 mixture of poly(hexamethyleneguanidinium chloride) and poly[2-(2-ethoxy)ethoxyethyl)guanidinium chloride] (cf. Antibiotika Monitor, volume 22, issue Jan. 2, 2006, online edition at http://www.antibiotikamonitor.at/06_{—}12/06_{—}12_inhalt.htm). However, this is actually a copolycondensate of guanidine hydrochloride and triethyleneglycoldiamine and hexamethylenediamine, the components being employed in a ratio of 4:1:3 (cf. WO 2006/047800).

Since the environmental and economic requirements imposed on modern-day crop protection compositions are continually increasing, with regard, for example, to the spectrum of action, toxicity, selectivity, application rate, formation of residues, and favourable preparability, and since, furthermore, there may be problems, for example, with resistances, a constant task is to develop new compositions which in some areas at least help to fulfil the abovementioned requirements.

The present invention provides a use which in some aspects at least achieves the stated objective. Moreover, active compound combinations are provided which likewise in some aspects at least achieve the stated objective.

Surprisingly, it has now been found that polymeric guanidine derivatives which are present in the form of their hydroxide (hereinbelow guanidinium hydroxides) are particularly suitable for the curative and/or preventive treatment of unwanted microorganisms such as bacteria and phytopathogenic fungi in crop protection including the treatment of seed.

The guanidinium hydroxides which can be used according to the invention are based on a diamine which contains oxyalkylene chains and/or alkylene groups inbetween two amino groups, which can be obtained by polycondensation of a guanidine acid addition salt with the diamine, giving a polycondensation product in salt form which is then converted by basic anion exchange into the hydroxide form.

A preferred embodiment of the guanidinium hydroxide used according to the invention comprises, as representatives of the group of the polyoxyalkyleneguanidine salts, those which are obtained using triethyleneglycoldiamine (relative molecular mass: 148), polyoxypropylenediamine (relative molecular mass: 230) and polyoxyethylenediamine (relative molecular mass: 600), or else polyhexamethylenediamine (relative molecular mass:.

A further preferred embodiment of the guanidinium hydroxide used according to the invention is characterized in that it comprises, as the polymeric guanidine derivative, poly[2-(2-ethoxyethoxyethyl)guanidinium hydroxide] having at least 3 guanidinium radicals.

Preference is furthermore given to using guanidinium hydroxides where the polycondensation product in salt form is obtainable beforehand by polycondensation of a guanidine acid addition salt with an alkylenediamine and an oxyalkylenediamine in a molar ratio of from 4:1 to 1:4 (alkylenediamine/oxyalkylenediamine), preferably from 3:1 to 1:3.

A preferred diamine is an alkylenediamine of the general formula

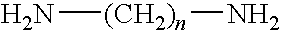

in which n is an integer from 2 to 10, in particular 6.

Furthermore, a preferred oxyalkylenediamine is a compound of the general formula

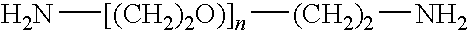

in which m is an integer from 2 to 5, in particular 2.

The mean molecular mass of the guanidinium hydroxide which can be used according to the invention is preferably in the range of from 500 to 3000, particularly preferably in the range of from 500 to 2000, very particularly preferably in the range of from 500 to 1500. Preference is furthermore given to a mean molecular mass of 1000.

The salts, in particular the hydrochlorides, which can be used as starting materials for the guanidinium hydroxides which can be used according to the invention are known (cf. WO 01/85676).

For example, the guanidinium hydroxide, which can be used according to the invention, poly[2-(2-ethoxyethoxyethyl)guanidinium hydroxide] having a mean molecular mass of about 1000 can be obtained by dissolving 4.43 mol of guanidinium hydrochloride in 4.03 mol of triethyleneglycoldiamine at 50° C. The solution is then heated to 120° C. and stirred at this temperature for 2 hours. The temperature is then maintained for 2 hours, a vacuum is then (0.1 bar) applied and the solution is stirred under reduced pressure at 170° C. for 2 more hours. The mixture is then vented to atmospheric pressure, cooled to 120° C. and diluted with demineralized water to about 50%. The mixture is neutralized to a pH of about 6 using phosphoric acid, allowed to cool and diluted to the desired concentration. The hydroxide form is then prepared by treating the solution obtained beforehand with a strongly alkaline anion exchanger in hydroxide form, expediently in an exchange column (for example “Ambersep 900 OH” or “Lewatit MP 500”). This gives the guanidinium hydroxide which can be used according to the invention and has the following characteristics: empirical formula: C₂₁H₅₁N₉O₅ or 49.5% carbon, 10% hydrogen, 24.8% nitrogen and 15.7% oxygen.

Analogously, a polycondensate is obtained from 1 mol of guanidine hydrochloride and a mixture of 0.75 mol of hexamethylenediamine and 0.25 mol of triethyleneglycoldiamine by mixing, at room temperature, 37 g (0.25 mol) of liquid triethyleneglycoldiamine (relative molecular mass 148) and 87 g (0.75 mol) of hexamethylenediamine (relative molecular mass 116) with 96.5 g (1 mol) of pulverulent guanidine hydrochloride (relative molecular mass 96.5). With constant mixing, the reaction material is heated at 140° C. for 1 hour and then maintained with stirring for one hour. With stirring, the mixture is then heated at a temperature of 170° C. for one hour and maintained at this temperature for a further hour. A vacuum is then applied carefully, and the solution is stirred under reduced pressure at 170° C. for another hour. The mixture is then vented to atmospheric pressure, cooled to 120° C. and diluted with demineralized water to about 50%. The mixture is neutralized to a pH of about 6 using phosphoric acid, allowed to cool and diluted to the desired concentration. The hydroxide form is then prepared by treating the solution obtained beforehand with a strongly alkaline anion exchanger in hydroxide form, expediently in an exchange column (for example “Ambersep 900 OH” or “Lewatit MP 500”). This gives the guanidinium hydroxide which can be used according to the invention.

The present invention furthermore relates to active compound combinations or compositions comprising at least one guanidinium hydroxide and at least one further fungicidally active compound.

Surprisingly, it has now been found that the active compound combinations or compositions according to the invention do not only exhibit an additive effect of the activity of the individual components, but a synergistic effect. Therefore, firstly, the customary application rate of the individual substances can be reduced. Secondly, the active compound combinations according to the invention offer a high degree of activity against phytopathogens even in cases where the individual compounds are employed in amounts where, for their part, they no longer show (sufficient) activity. In principle, this allows, firstly, the activity spectrum to be broadened and, secondly, better safety during handling.

In addition to the fungicidal synergistic activity, the active compound combinations according to the invention have further surprising properties which, in a wider sense, may also be called synergistic, such as, for example: broadening of the activity spectrum, for example to resistant pathogens of plant diseases; lower application rates of the active compounds; sufficient control of pests with the aid of the active compound combinations according to the invention even at application rates where the individual compounds show no or virtually no activity; advantageous behaviour during formulation or during use, for example during grinding, sieving, emulsifying, dissolving or application; improved storage stability and light stability; advantageous residue formation; improved toxicological or ecotoxicological behaviour; improved properties of the plant, for example better growth, increased harvest yields, a better developed root system, a larger leaf area, greener leaves, stronger shoots, less seed required, lower phytotoxicity, mobilization of the defense system of the plant, good compatibility with plants. Thus, the use of the active compound combinations or compositions according to the invention contributes considerably to keeping young cereal stands healthy, which increases, for example, the winter survival of the cereal seed treated, and also safeguards quality and yield. Moreover, the active compound combinations according to the invention may contribute to enhanced systemic action. Even if the individual compounds of the combination have no sufficient systemic properties, the active compound combinations according to the invention may still have this property. In a similar manner, the active compound combinations according to the invention may result in higher persistency of the fungicidal action.

The guanidinium hydroxide (component A) employed is preferably poly[2-(2-ethoxyethoxyethyl)guanidinium hydroxide] having a mean molecular mass of about 1000 (A-1), a mean molecular mass of about 1500 (A-2), a mean molecular mass of about 500 (A-3), a mean molecular mass of about 2000 (A-4), a mean molecular mass of about 2500 (A-5) or a mean molecular mass of about 3000 (A-6), and moreover a polycondensate of poly(hexamethyleneguanidinium hydroxide) and poly[2-(2-ethoxy)ethoxyethyl)guanidinium hydroxide] in a molar ratio of 3:1 (A-7) [where the copolycondensate (A-7) is better described by the starting materials guanidine hydrochloride, triethyleneglycoldiamine and hexamethylenediamine, employed in a ratio of 4:1:3, which after polymerization are converted into the hydroxide].

The fungicidal mixing partner is selected from the group consisting of

(B) inhibitors (or salts thereof) of ergosterol biosynthesis,
preferably selected from the group consisting of (B-1) aldimorph (1704-28-5), (B-2) azaconazole (60207-31-0), (B-3) bitertanol (55179-31-2), (B-4) bromuconazole (116255-48-2), (B-5) cyproconazole (113096-99-4), (B-6) diclobutrazole (75736-33-3), (B-7) difenoconazole (119446-68-3), (B-8) diniconazole (83657-24-3), (B-9) diniconazole-M (83657-18-5), (B-10) dodemorph (1593-77-7), (B-11) dodemorph acetate (31717-87-0), (B-12) epoxiconazole (106325-08-0), (B-13) etaconazole (60207-93-4), (B-14) fenarimol (60168-88-9), (B-15) fenbuconazole (114369-43-6), (B-16) fenhexamid (126833-17-8), (B-17) fenpropidin (67306-00-7), (B-18) fenpropimorph (67306-03-0), (B-19) fluquinconazole (136426-54-5), (B-20) flurprimidol (56425-91-3), (B-21) flusilazole (85509-19-9), (B-22) flutriafol (76674-21-0), (B-23) furconazole (112839-33-5), (B-24) furconazole-cis (112839-32-4), (B-25) hexaconazole (79983-71-4), (B-26) imazalil (60534-80-7), (B-27) imazalil sulphate (58594-72-2), (B-28) imibenconazole (86598-92-7), (B-29) ipconazole (125225-28-7), (B-30) metconazole (125116-23-6), (B-31) myclobutanil (88671-89-0), (B-32) naftifine (65472-88-0), (B-33) nuarimol (63284-71-9), (B-34) oxpoconazole (174212-12-5), (B-35) paclobutrazole (76738-62-0), (B-36) pefurazoate (101903-30-4), (B-37) penconazole (66246-88-6), (B-38) piperalin (3478-94-2), (B-39) prochloraz (67747-09-5), (B-40) propiconazole (60207-90-1), (B-41) prothioconazole (178928-70-6), (B-42) pyributicarb (88678-67-5), (B-43) pyrifenox (88283-41-4), (B-44) quinconazole (13970-75-8), (B-45) simeconazole (149508-90-7), (B-46) spiroxamine (118134-30-8), (B-47) tebuconazole (107534-96-3), (B-48) terbinafine (91161-71-6), (B-49) tetraconazole (112281-77-3), (B-50) triadimefon (43121-43-3), (B-51) triadimenol (89482-17-7), (B-52) tridemorph (81412-43-3), (B-53) triflumizole (68694-11-1), (B-54) triforine (26644-46-2), (B-55) triticonazole (131983-72-7), (B-56) uniconazole (83657-22-1), (B-57) viniconazole (77174-66-4), (B-58) voriconazole (137234-62-9), (B-59) 1-(4-chlorophenyl)-2-(1H-1,2,4-triazol-1-yl)cycloheptanol (129586-32-9), (B-60) methyl 1-(2,2-dimethyl-2,3-dihydro-1H-inden-1-yl)-1H-imidazole-5-carboxylate (11323-95-0), (B-61) 0-{1-[(4-methoxyphenoxy)methyl]-2,2-dimethylpropyl}1H-imidazole-1-carbothioate (111226-71-2),
particularly preferably selected from the group consisting of (B-3) bitertanol, (B-5) cyproconazole, (B-12) epoxiconazole, (B-17) fenpropidin, (B-18) fenpropimorph, (B-19) fluquinconazole, (B-30) metconazole, (B-41) prothioconazole, (B-46) spiroxamine, (B-47) tebuconazole, (B-51) triadimenol; also particularly preferably selected from the group consisting of (B-3) bitertanol, (B-7) difenoconazole, (B-12) epoxiconazole, (B-16) fenhexamid, (B-26) imazalil, (B-39) prochloraz, (B-41) prothioconazole, (B-46) spiroxamine, (B-47) tebuconazole, (B-52) tridemorph;
or
(C) respiratory chain inhibitors (or salts thereof) at complex I or II,
preferably selected from the group consisting of (C-1) diflumetorim (130339-07-0), (C-2) bixafen[N-(3′,4′-dichloro-5-fluoro-1,1′-biphenyl-2-yl)-3-(difluoromethyl)-1-methyl-1H-pyrazole-4-carboxamide], (C-3) boscalid (188425-85-6), (C-4) carboxin (5234-68-4), (C-5) fenfuram (24691-80-3), (C-6) fluopyram {N-{2-[3-chloro-5-(trifluoromethyl)pyridin-2-yl]ethyl}-2-(trifluoromethyl)benzamide)}, (C-7) flutolanil (66332-96-5), (C-8) furametpyr (123572-88-3), (C-9) furmecyclox (60568-05-0), (C-10) mepronil (55814-41-0), (C-11) oxycarboxin (5259-88-1), (C-12) penthiopyrad (183675-82-3), (C-13) thifluzamide (130000-40-7), (C-14) N-[2-(1,3-dimethylbutyl)phenyl]-5-fluoro-1,3-dimethyl-1H-pyrazole-4-carboxamide (known from WO 03/010149), (C-15) N-{2-[1,1′-bi(cyclopropyl)-2-yl]phenyl}-3-(difluoromethyl)-1-methyl-1H-pyrazole-4-carboxamide, (C-16) 3-(difluoromethyl)-N-[(9R)-9-isopropyl-1,2,3,4-tetrahydro-1,4-methanonaphthalen-5-yl]-1-methyl-1H-pyrazole-4-carboxamide, (C-17) 3-(difluoromethyl)-N-[(9S)-9-isopropyl-1,2,3,4-tetrahydro-1,4-methanonaphthalen-5-yl]-1-methyl-1H-pyrazole-4-carboxamide, (C-18) 1-methyl-N-[2-(1,1,2,2-tetrafluoroethoxy)phenyl]-3-(trifluoromethyl)-1H-pyrazole-4-carboxamide, (C-19) 3-(difluoromethyl)-1-methyl-N-[2-(1,1,2,2-tetrafluoroethoxy)phenyl]-1H-pyrazole-4-carboxamide, (C-20) 1-methyl-3-(trifluoromethyl)-N-[2′-(trifluoromethyl)biphenyl-2-yl]-1H-pyrazole-4-carboxamide, (C-21) N-(4′-chlorobiphenyl-2-yl)-3-(difluoromethyl)-1-methyl-1H-pyrazole-4-carboxamide, (C-22) N-(2′,4′-dichlorobiphenyl-2-yl)-3-(difluoromethyl)-1-methyl-1H-pyrazole-4-carboxamide, (C-23) 3-(difluoromethyl)-1-methyl-N-[4′-(trifluoromethyl)biphenyl-2-yl]-1H-pyrazole-4-carboxamide, (C-24) N-(2′,5′-difluorobiphenyl-2-yl)-1-methyl-3-(trifluoromethyl)-1H-pyrazole-4-carboxamide, (C-25) 3-(difluoromethyl)-N-[4′-(3,3-dimethylbut-1-yn-1-yl)biphenyl-2-yl]-1-methyl-1H-pyrazole-4-carboxamide (known from WO 04/058723), (C-26) 3-(difluoromethyl)-1-methyl-N-(4′-prop-1-yn-1-ylbiphenyl-2-yl)-1H-pyrazole-4-carboxamide (known from WO 04/058723), (C-27) 5-fluoro-1,3-dimethyl-N-(4′-prop-1-yn-1-ylbiphenyl-2-yl)-1H-pyrazole-4-carboxamide (known from WO 04/058723), (C-28) 2-chloro-N-(4′-prop-1-yn-1-ylbiphenyl-2-yl)nicotinamide (known from WO 04/058723), (C-29) N-[4′-(ethynylbiphenyl-2-yl]-3-(difluoromethyl)-1-methyl-1H-pyrazole-4-carboxamide (known from WO 04/058723), (C-30) N-(4′-ethynylbiphenyl-2-yl)-5-fluoro-1,3-dimethyl-1H-pyrazole-4-carboxamide (known from WO 04/058723), (C-31) 2-chloro-N-(4′-ethynylbiphenyl-2-yl)nicotinamide (known from WO 04/058723), (C-32) 3-(difluoromethyl)-N-[4′-(3,3-dimethylbut-1-yn-1-yl)biphenyl-2-yl]-1-methyl-1H-pyrazole-4-carboxamide (known from WO 04/058723), (C-33) N-[4′-(3,3-dimethylbut-1-yn-1-yl)biphenyl-2-yl]-5-fluoro-1,3-dimethyl-1H-pyrazole-4-carboxamide (known from WO 04/058723), (C-34) 2-chloro-N-[4′-(3,3-dimethylbut-1-yn-1-yl)biphenyl-2-yl]nicotinamide (known from WO 04/058723), (C-35) 4-(difluoromethyl)-2-methyl-N-[4′-(trifluoromethyl)-1,1′-biphenyl-2-yl]-1,3-thiazole-5-carboxamide (known from WO 04/058723), (C-36) 5-fluoro-N-[4′-(3-hydroxy-3-methylbut-1-yn-1-yl)biphenyl-2-yl]-1,3-dimethyl-1H-pyrazole-4-carboxamide (known from WO 04/058723), (C-37) 2-chloro-N-[4′-(3-hydroxy-3-methylbut-1-yn-1-yl)biphenyl-2-yl]nicotinamide (known from WO 04/058723), (C-38) 3-difluoromethyl-N-[4′-(3-methoxy-3-methylbut-1-yn-1-yl)biphenyl-2-yl]-1-methyl-1H-pyrazole-4-carboxamide (known from WO 04/058723), (C-39) 5-fluoro-N-[4′-(3-methoxy-3-methylbut-1-yn-1-yl)biphenyl-2-yl]-1,3-dimethyl-1H-pyrazole-4-carboxamide (known from WO 04/058723), (C-40) 2-chloro-N-[4′-(3-methoxy-3-methylbut-1-yn-1-yl)biphenyl-2-yl]nicotinamide (known from WO 04/058723), (C-41) isopyrazam {3-(difluoromethyl)-1-methyl-N-[1,2,3,4-tetrahydro-9-(1-methylethyl)-1,4-methanonaphthalen-5-yl]-1H-pyrazole-4-carboxamide mixture of 2 syn-isomers and 2 anti-isomers (683777-13-1 and 683777-14-2)},
preferably selected from the group consisting of (C-2) bixafen, (C-3) boscalid, (C-4) carboxin, (C-6) fluopyram, (C-12) penthiopyrad, (C-14) N-[2-(1,3-dimethylbutyl)phenyl]-5-fluoro-1,3-dimethyl-1H-pyrazole-4-carboxamide, (C-15) N-{2-[1,1′-bi(cyclopropyl)-2-yl]phenyl}-3-(difluoromethyl)-1-methyl-1H-pyrazole-4-carboxamide, (C-16) 3-(difluoromethyl)-N-[(9R)-9-isopropyl-1,2,3,4-tetrahydro-1,4-methanonaphthalen-5-yl]-1-methyl-1H-pyrazole-4-carboxamide, (C-17) 3-(difluoromethyl)-N-[(9S)-9-isopropyl-1,2,3,4-tetrahydro-1,4-methanonaphthalen-5-yl]-1-methyl-1H-pyrazole-4-carboxamide, (C-19) 3-(difluoromethyl)-1-methyl-N-[2-(1,1,2,2-tetrafluoroethoxy)phenyl]-1H-pyrazole-4-carboxamide, (C-25) 3-(difluoromethyl)-N-[4′-(3,3-dimethylbut-1-yn-1-yl)biphenyl-2-yl]-1-methyl-1H-pyrazole-4-carboxamide, (C-41) isopyrazam;
very particularly preferably selected from the group consisting of (C-2) bixafen, (C-3) boscalid, (C-4) carboxin, (C-6) fluopyram, (C-12) penthiopyrad, (C-14) N-[2-(1,3-dimethylbutyl)phenyl]-5-fluoro-1,3-dimethyl-1H-pyrazole-4-carboxamide, (C-19) 3-(difluoromethyl)-1-methyl-N-[2-(1,1,2,2-tetrafluoroethoxy)phenyl]-1H-pyrazole-4-carboxamide, (C-41) isopyrazam;
or
(D) respiratory chain inhibitors (or salts thereof) at complex III,
preferably selected from the group consisting of (D-1) famoxadone (131807-57-3), (D-2) fenamidone (161326-34-7), (D-3) amisulbrom (348635-87-0), (D-4) azoxystrobin (131860-33-8), (D-5) cyazofamid (120116-88-3), (D-6) dimoxystrobin (141600-52-4), (D-7) enestrobin (238410-11-2), (D-8) fluoxastrobin (361377-29-9), (D-9) kresoxim-methyl (143390-89-0), (D-10) metominostrobin (133408-50-1), (D-11) orysastrobin (189892-69-1), (D-12) picoxystrobin (117428-22-5), (D-13) pyraclostrobin (175013-18-0), (D-14) pyribencarb (799247-52-2), (D-15) trifloxystrobin (141517-21-7), (D-16) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (D-17) (2E)-2-(2-{[6-(3-chloro-2-methylphenoxy)-5-fluoro-4-pyrimidinyl]oxy}phenyl)-2-(methoxyimino)-N-methylethanamide, (D-18) 2-chloro-N-(2,3-dihydro-1,1,3-trimethyl-1H-inden-4-yl)-3-pyridinecarboxamide (119899-14-8), (D-19) (2E)-2-(methoxyimino)-N-methyl-2-(2-{[({(1E)-1-[3-(trifluoromethyl)phenyl]ethylidene}amino)oxy]methyl}phenyl)ethanamide, (D-20) N-(3-ethyl-3,5,5-trimethylcyclohexyl)-3-(formylamino)-2-hydroxybenzamide (226551-21-9), (D-21) (2E)-2-(methoxyimino)-N-methyl-2-{2-[(E)-({1-[3-(trifluoromethyl)phenyl]ethoxy}-imino)methyl]phenyl}ethanamide, (D-22) (2E)-2-{2-[({[(1E)-1-(3-{[(E)-1-fluoro-2-phenylvinyl]oxy}-phenyl)ethylidene]aminooxy)methyl]phenyl}-2-(methoxyimino)-N-methylacetamide (326896-28-0) particularly preferably selected from the group consisting of (D-4) azoxystrobin, (D-6) dimoxystrobin, (D-8) fluoxastrobin, (D-9) kresoxim-methyl, (D-10) metominostrobin, (D-15) trifloxystrobin, (D-11) orysastrobin, (D-12) picoxystrobin, (D-13) pyraclostrobin;
also particularly preferably selected from the group consisting of (D-2) fenamidone, (D-4) azoxystrobin, (D-8) fluoxastrobin, (D-13) pyraclostrobin, (D-15) trifloxystrobin;
or
E) inhibitors (or salts thereof) of mitosis and cell division,
preferably selected from the group consisting of
(E-1) benomyl (17804-35-2), (E-2) carbendazim (10605-21-7), (E-3) chlorfenazole (3574-96-7), (E-4) diethofencarb (87130-20-9), (E-5) ethaboxam (162650-77-3), (E-6) fluopicolide (239110-15-7), (E-7) fuberidazole (3878-19-1), (E-8) pencycuron (66063-05-6), (E-9) profenofos (41198-08-7), (E-10) thiabendazole (148-79-8), (E-11) thiophanate (23564-06-9), (E-12) thiophanate-methyl (23564-05-8), (E-13) zoxamide (156052-68-5), (E-14) 5-chloro-6-(2,4,6-trifluorophenyl)-7-(4-methylpiperidin-1-yl)[1,2,4]triazolo[1,5-a]pyrimidine (214706-53-3),
particularly preferably selected from the group consisting of (E-2) carbendazim, (E-6) fluopicolide, (E-8) pencycuron, (E-10) thiabendazole, (E-11) thiophanate, (E-12) thiophanate-methyl, (E-13) zoxamide, (E-14) 5-chloro-6-(2,4,6-trifluorophenyl)-7-(4-methylpiperidin-1-yl)[1,2,4]triazolo[1,5-a]pyrimidine;
very particularly preferably (E-6) fluopicolide;
or
(F) multisite active compounds (or salts thereof),
preferably selected from the group consisting of
(F-1) Bordeaux mixture (8011-63-0), (F-2) captafol (2425-06-1), (F-3) captan (133-06-2), (F-4) chlorothalonil (1897-45-6), (F-5) copper hydroxide (20427-59-2), (F-6) copper naphthenate (1338-02-9), (F-7) copper oxide (1317-39-1), (F-8) copper oxychloride (1332-40-7), (F-9) copper sulphate (7758-98-7), (F-10) dichlofluanid (1085-98-9), (F-11) dithianon (3347-22-6), (F-12) dodine (2439-10-3), (F-13) ferbam (14484-64-1), (F-14) fluorofolpet (719-96-0), (F-15) folpet (133-07-3), (F-16) guazatine (108173-90-6), (F-17) iminoctadine (13516-27-3), (F-18) iminoctadine triacetate (57520-17-9), (F-19) mancopper (53988-93-5), (F-20) mancozeb (8018-01-7), (F-21) maneb (12427-38-2), (F-22) metiram (9006-42-2), (F-23) oxine-copper (10380-28-6), (F-24) propineb (12071-83-9), (F-25) sulphur and sulphur preparations including calcium polysulphide, (F-26) thiram (137-26-8), (F-27) tolylfluanid (731-27-1), (F-28) zineb (12122-67-7), (F-29) ziram (137-30-4);
particularly preferably selected from the group consisting of (F-4) chorothalonil, (F-10) dichlofluanid, (F-12) dodine, (F-14) fluorofolpet, (F-15) folpet, (F-16) guazatine, (F-17) iminoctadine, (F-18) iminoctadine triacetate, (F-20) mancozeb, (F-21) maneb, (F-22) metiram, (F-24) propineb, (F-26) thiram, (F-27) tolylfluanid, (F-28) zineb, (F-29) ziram;
very particularly preferably selected from the group consisting of (F-4) chorothalonil, (F-20) mancozeb, (F-24) propineb;
or
(G) the other fungicides (or salts thereof) below,
preferably selected from the group consisting of (G-1) acibenzolar-5-methyl (135158-54-2), (G-2) isotianil (224049-04-1), (G-3) probenazole (27605-76-1), (G-4) tiadinil (223580-51-6), (G-5) andoprim (23951-85-1), (G-6) blasticidin-S (2079-00-7), (G-7) cyprodinyl (121552-61-2), (G-8) kasugamycin (6980-18-3), (G-9) mepanipyrim (110235-47-7), (G-10) pyrimethanil (53112-28-0), (G-11) fentin acetate (900-95-8), (G-12) fentin chloride (639-58-7), (G-13) fentin hydroxide (76-87-9), (G-14) silthiofam (175217-20-6), (G-15) benthiavalicarb (177406-68-7), (G-16) dimethomorph (110488-70-5), (G-17) flumorph (211867-47-9), (G-18) iprovalicarb (140923-17-7), (G-19) mandipropamid (374726-62-2), (G-20) valiphenal (283159-94-4), (G-21) polyoxins (11113-80-7), (G-22) polyoxorim (22976-86-9), (G-23) validamycin A (37248-47-8), (G-24) biphenyl (92-52-4), (G-25) chloroneb (2675-77-6), (G-26) chlozolinate (84332-86-5), (G-27) edifenfos (17109-49-8), (G-28) etridiazole (2593-15-9), (G-29) iodocarb (55406-53-6), (G-30) iprobenfos (26087-47-8), (G-31) iprodione (36734-19-7), (G-32) isoprothiolane (50512-35-1), (G-33) procymidone (32809-16-8), (G-34) propamocarb (25606-41-1), (G-35) propamocarb hydrochloride (25606-41-1), (G-36) prothiocarb (19622-08-3), (G-37) pyrazophos (13457-18-6), (G-38) tolcofos-methyl (57018-04-9), (G-39) vinclozolin (50471-44-8), (G-40) carpropamid (104030-54-8), (G-41) diclocymet (139920-32-4), (G-42) fenoxanil (115852-48-7), (G-43) phthalide (27355-22-2), (G-44) pyroquilon (57369-32-1), (G-45) tricyclazole (41814-78-2), (G-46) benalaxyl (71626-11-4), (G-47) benalaxyl-M (98243-83-5), (G-48) bupirimate (41483-43-6), (G-49) clozylacon (67932-85-8), (G-50) dimethirimol (5221-53-4), (G-51) ethirimol (23947-60-6), (G-52) furalaxyl (57646-30-7), (G-53) hymexazole (10004-44-1), (G-54) metalaxyl (57837-19-1), (G-55) metalaxyl-M (Mefenoxam) (70630-17-0), (G-56) ofurace (58810-48-3), (G-57) oxadixyl (77732-09-3), (G-58) oxolinic acid (14698-29-4), (G-59) fenpiclonil (74738-17-3), (G-60) fludioxonil (131341-86-1), (G-61) quinoxyfen (124495-18-7), (G-62) binapacryl (485-31-4), (G-63) dinocap (131-72-6), (G-64) fluazinam (79622-59-6), (G-65) meptyldinocap (131-72-6), (G-66) benthiazole (21564-17-0), (G-67) bethoxazin (163269-30-5), (G-68) capsimycin (70694-08-5), (G-69) carvone (99-49-0), (G-70) chinomethionat (2439-01-2), (G-71) cufraneb (11096-18-7), (G-72) cyflufenamid (180409-60-3), (G-73) cymoxanil (57966-95-7), (G-74) cyprosulfamide (221-667-31-8), (G-75) dazomet (533-74-4), (G-76) debacarb (62732-91-6), (G-77) dichlorophen (97-23-4), (G-78) diclomezine (62865-36-5), (G-79) dicloran (99-30-9), (G-80) difenzoquat (43222-48-6), (G-81) diphenylamine (122-39-4), (G-82) ferimzone (89269-64-7), (G-83) flumetover (154025-04-4), (G-84) fluoroimide (41205-21-4), (G-85) flusulfamide (106917-52-6), (G-86) fosetyl-Al (39148-24-8), (G-87) fosetyl-calcium, (G-88) fosetyl-sodium, (G-89) hexachlorobenzene (118-74-1), (G-90) irumamycin (81604-73-1), (G-91) methasulfocarb (66952-49-6), (G-92) methyl isothiocyanate (556-61-6), (G-93) metrafenone (220899-03-6), (G-94) mildiomycin (67527-71-3), (G-95) natamycin (7681-93-8), (G-96) nickel dimethyldithiocarbamate, (G-97) nitrothal-isopropyl (10552-74-6), (G-98) octhilinone (26530-20-1), (G-99) oxyfenthiin (34407-87-9), (G-100) pentachlorophenol (87-86-5), (G-101) phosphorous acid (13598-36-2), (G-102) propamocarb-fosetyl, (G-103) propanosin-sodium (88498-02-6), (G-104) proquinazid (189278-12-4), (G-105) pyrrolnitrins (1018-71-9), (G-106) quintozene (82-68-8), (G-107) tecloftalam (76280-91-6), (G-108) tecnazene (117-18-0), (G-109) triazoxide (72459-58-6), (G-110) trichlamide (70193-21-4), (G-111) zarilamid (84527-51-5), (G-112) 8-hydroxyquinoline sulphate (134-31-6), (G-113) 2,3,5,6-tetrachloro-4-(methylsulphonyl)pyridine (13108-52-6), (G-114) 3,4,5-trichloropyridine-2,6-dicarbonitrile (17824-85-0), (G-115) 3-[5-(4-chlorophenyl)-2,3-dimethylisoxazolidin-3-yl]pyridine, (G-116) N-(4-chloro-2-nitrophenyl)-N-ethyl-4-methylbenzenesulphonamide (304911-98-6), (G-117) 2,3-dibutyl-6-chlorothieno[2,3-d]pyrimidin-4(3H) one (221451-58-7), (G-118) 2-butoxy-6-iodo-3-propylbenzopyran-4-one, (G-119) N-(4-chlorobenzyl)-3-[3-methoxy-4-(prop-2-yn-1-yloxy)phenyl]propanamide, (G-120) N-(6-methoxy-3-pyridinyl)cyclopropanecarboxamide (112860-04-5), (G-121) N-[(4-chlorophenyl)(cyano)methyl]-3-[3-methoxy-4-(prop-2-yn-1-yloxy)phenyl]propanamide, (G-122) N-[(5-bromo-3-chloropyridin-2-yl)methyl]-2,4-dichloropyridine-3-carboxamide, (G-123) N-[1-(5-bromo-3-chloropyridin-2-yl)ethyl]-2,4-dichloropyridine-3-carboxamide, (G-124) N-[1-(5-bromo-3-chloropyridin-2-yl)ethyl]-2-fluoro-4-iodopyridine-3-carboxamide, (G-125) N′-[4-(3-tert-butyl-4-chlorophenoxy)-2,5-dimethylphenyl]-N-ethyl-N-methylimidoformamide, (G-126) N′-[4-(3-tert-butyl-4-chlorophenoxy)-2-methyl-5-(trifluoromethyl)phenyl]-N-ethyl-N-methylimidoformamide, (G-127) N′-[4-(3-tert-butyl-4-chlorophenoxy)-5-(difluoromethyl)-2-methylphenyl]-N-ethyl-N-methylimidoformamide, (G-128) N′-[4-(3-tert-butyl-4-fluorophenoxy)-2,5-dimethylphenyl]-N-ethyl-N-ethylimidoformamide, (G-129) N′-[4-(3-tert-butyl-4-fluorophenoxy)-2-methyl-5-(trifluoromethyl)phenyl]-N-ethyl-N-methylimidoformamide, (G-130) N′-[4-(3-tert-butyl-4-fluorophenoxy)-5-(difluoromethyl)-2-methylphenyl]-N-ethyl-N-methylimidoformamide, (G-131) N′-[4-(4-chloro-3-isopropylphenoxy)-2,5-dimethylphenyl]-N-ethyl-N-methylimidoformamide, (G-132) N′-[4-(4-chloro-3-isopropylphenoxy)-2-methyl-5-(trifluoromethyl)phenyl]-N-ethyl-N-methylimidoformamide, (G-133) N′-[4-(4-chloro-3-isopropylphenoxy)-5-(difluoromethyl)-2-methylphenyl]-N-ethyl-N-methylimidoformamide, (G-134) N′-{4-[3-tert-butyl-1,2,4-thiadiazol-5-yl)oxy]-2,5-dimethylphenyl}-N-ethyl-N-methylimidoformamide, (G-135) N′-{4-[(3-tert-butyl-1,2,4-thiadiazol-5-yl)oxy]-2-methyl-5-(trifluoromethyl)phenyl}-N-ethyl-N-methylimidoformamide, (G-136) N′-{4-[(4-tert-butyl-1,3-thiazol-2-yl)oxy]-2,5-dimethylphenyl}-N-ethyl-N-methylimidoformamide, (G-137) N′-{4-[(4-tert-butyl-1,3-thiazol-2-yl)oxy]-2-methyl-5-(trifluoromethyl)phenyl}-N-ethyl-N-methylimidoformamide, (G-138) N′-{5-(difluoromethyl)-2-methyl-4-[3-(trimethylsilyl)propoxy]-phenyl}-N-ethyl-N-methylimidoformamide, (G-139) N-ethyl-N′-[4-(4-fluoro-3-isopropylphenoxy)-2,5-dimethylphenyl]-N-ethylimidoformamide, (G-140) N-ethyl-N′-[4-(4-fluoro-3-isopropylphenoxy)-2-methyl-5-(trifluoromethyl)phenyl]-N-methylimidoformamide, (G-141) N-ethyl-N′-[4-(4-fluoro-3-isopropylphenoxy)-2-methyl-5-(trifluoromethyl)phenyl]-N-methylimidoformamide, (G-142) N-ethyl-N′-{4-[3-isopropyl-1,2,4-thiadiazol-5-yl)oxy]-2,5-dimethylphenyl}-N-methylimidoformamide, (G-143) N-ethyl-N′-{4-[(3-isopropyl-1,2,4-thiadiazol-5-yl)oxy]-2-methyl-5-(trifluoromethyl)phenyl}-N-methylimidoformamide, (G-144) N-ethyl-N′-{4-[(4-isopropyl-1,3-thiazol-2-yl)oxy]-2,5-dimethylphenyl}-N-methylimidoformamide, (G-145) N-ethyl-N′-{4-[(4-isopropyl-1,3-thiazol-2-yl)oxy]-2-methyl-5-(trifluoromethyl)phenyl}-N-methylimidoformamide, (G-146) N-ethyl-N-methyl-N′-{2-methyl-5-(trifluoromethyl)-4-[3-(trimethylsilyl)propoxy]phenyl}imidoformamide, (G-147) S-allyl-5-amino-2-isopropyl-4-(2-methylphenyl)-3-oxo-2,3-dihydro-1H-pyrazole-1-carbothioate;
particularly preferably selected from the group consisting of (G-1) acibenzolar-5-methyl, (G-46) benalaxyl, (G-47) benalaxyl-M, (G-15) benthiavalicarb, (G-73) cymoxanil, (G-7) cyprodinyl, (G-74) cyprosulfamide, (G-16) dimethomorph, (G-59) fenpiclonil, (G-64) fluazinam, (G-60) fludioxonil, (G-86) fosetyl-Al, (G-52) furalaxyl, (G-31) iprodione, (G-18) iprovalicarb, (G-2) isotianil, (G-19) mandipropamid, (G-9) mepanipyrim, (G-54) metalaxyl, (G-55) metalaxyl-M, (G-93) metrafenone, (G-138) N′-{5-(difluoromethyl)-2-methyl-4-[3-(trimethylsilyl)propoxy]phenyl}-N-ethyl-N-methylimidoformamide, (G-146) N-ethyl-N-methyl-N′-{2-methyl-5-(trifluoromethyl)-4-[3-(trimethylsilyl)propoxy]phenyl}-imidoformamide, (G-57) oxadixyl, (G-101) phosphorous acid, (G-33) procymidone, (G-34) propamocarb, (G-35) propamocarb hydrochloride, (G-10) pyrimethanil, (G-61) quinoxyfen, (G-14) silthiofam, (G-20) valiphenal, (G-39) vinclozolin;
very particularly preferably selected from the group consisting of (G-15) benthiavalicarb, (G-73) cymoxanil, (G-16) dimethomorph, (G-64) fluazinam, (G-60) fludioxonil, (G-86) fosetyl-Al, (G-31) iprodione, (G-18) iprovalicarb, (G-55) metalaxyl-M, (G-101) phosphorous acid, (G-35) propamocarb hydrochloride, (G-10) pyrimethanil.

Preference is given to the active compound combinations listed below and to compositions comprising these active compound combinations:

Poly[2-(2-ethoxyethoxyethyl)guanidinium hydroxide] having a mean molecular mass of about 1000 (A-1) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Poly[2-(2-ethoxyethoxyethyl)guanidinium hydroxide] having a mean molecular mass of about 1500 (A-2) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Poly[2-(2-ethoxyethoxyethyl)guanidinium hydroxide] having a mean molecular mass of about 500 (A-3) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Poly[2-(2-ethoxyethoxyethyl)guanidinium hydroxide] having a mean molecular mass of about 2000 (A-4) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Poly[2-(2-ethoxyethoxyethyl)guanidinium hydroxide] having a mean molecular mass of about 2500 (A-5) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Poly[2-(2-ethoxyethoxyethyl)guanidinium hydroxide] having a mean molecular mass of about 3000 (A-6) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Polycondensate of poly(hexamethyleneguanidinium hydroxide) and poly[2-(2-ethoxy)ethoxyethyl)guanidinium hydroxide] in a molar ratio of 3:1 (A-7) and at least one further active compound selected from the group consisting of (B-1), (B-2), (B-3), (B-4), (B-5), (B-6), (B-7), (B-8), (B-9), (B-10), (B-11), (B-12), (B-13), (B-14), (B-15), (B-16), (B-17), (B-18), (B-19), (B-20), (B-21), (B-22), (B-23), (B-24), (B-25), (B-26), (B-27), (B-28), (B-29), (B-30), (B-31), (B-32), (B-33), (B-34), (B-35), (B-36), (B-37), (B-38), (B-39), (B-40), (B-41), (B-42), (B-43), (B-44), (B-45), (B-46), (B-47), (B-48), (B-49), (B-50), (B-51), (C-1), (C-2), (C-3), (C-4), (C-5), (C-6), (C-7), (C-8), (C-9), (C-10), (C-11), (C-12), (C-13), (C-14), (C-15), (C-16), (C-17), (C-18), (C-19), (C-20), (C-21), (C-22), (C-23), (C-24), (C-25), (C-26), (C-27), (C-28), (C-29), (C-30), (C-31), (C-32), (C-33), (C-34), (C-35), (C-36), (C-37), (C-38), (C-39), (C-40), (D-1), (D-2), (D-3), (D-4), (D-5), (D-6), (D-7), (D-8), (D-9), (D-10), (D-11), (D-12), (D-13), (D-14), (D-15), (D-16), (D-17), (D-18), (D-19), (D-20), (D-21), (D-22), (E-1), (E-2), (E-3), (E-4), (E-5), (E-6), (E-7), (E-8), (E-9), (E-10), (E-11), (E-12), (E-13), (E-14), (F-1), (F-2), (F-3), (F-4), (F-5), (F-6), (F-7), (F-8), (F-9), (F-10), (F-11), (F-12), (F-13), (F-14), (F-15), (F-16), (F-17), (F-18), (F-19), (F-20), (F-21), (F-22), (F-23), (F-24), (F-25), (F-26), (F-27), (F-28), (F-29), (G-1), (G-2), (G-3), (G-4), (G-5), (G-6), (G-7), (G-8), (G-9), (G-10), (G-11), (G-12), (G-13), (G-14), (G-15), (G-16), (G-17), (G-18), (G-19), (G-20), (G-21), (G-22), (G-23), (G-24), (G-25), (G-26), (G-27), (G-28), (G-29), (G-30), (G-31), (G-32), (G-33), (G-34), (G-35), (G-36), (G-37), (G-38), (G-39), (G-40), (G-41), (G-42), (G-43), (G-44), (G-45), (G-46), (G-47), (G-48), (G-49), (G-50), (G-51), (G-52), (G-53), (G-54), (G-55), (G-56), (G-57), (G-58), (G-59), (G-60), (G-61), (G-62), (G-63), (G-64), (G-65), (G-66), (G-67), (G-68), (G-69), (G-70), (G-71), (G-72), (G-73), (G-74), (G-75), (G-76), (G-77), (G-78), (G-79), (G-80), (G-81), (G-82), (G-83), (G-84), (G-85), (G-86), (G-87), (G-88), (G-89), (G-90), (G-91), (G-92), (G-93), (G-94), (G-95), (G-96), (G-97), (G-98), (G-99), (G-100), (G-101), (G-102), (G-103), (G-104), (G-105), (G-106), (G-107), (G-108), (G-109), (G-110), (G-111), (G-112), (G-113), (G-114), (G-115), (G-116), (G-117), (G-118), (G-119), (G-120), (G-121), (G-122), (G-123), (G-124), (G-125), (G-126), (G-127), (G-128), (G-129), (G-130), (G-131), (G-132), (G-133), (G-134), (G-135), (G-136), (G-137), (G-138), (G-139), (G-140), (G-141), (G-142), (G-143), (G-144), (G-145), (G-146), (G-147).

Particular preference is given to the active compound combinations listed below and to compositions comprising these active compound combinations:

Poly[2-(2-ethoxyethoxyethyl)guanidinium hydroxide] having a mean molecular mass of about 1000 (A-1) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Poly[2-(2-ethoxyethoxyethyl)guanidinium hydroxide] having a mean molecular mass of about 1500 (A-2) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Poly[2-(2-ethoxyethoxyethyl)guanidinium hydroxide] having a mean molecular mass of about 500 (A-3) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Poly[2-(2-ethoxyethoxyethyl)guanidinium hydroxide] having a mean molecular mass of about 2000 (A-4) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Poly[2-(2-ethoxyethoxyethyl)guanidinium hydroxide] having a mean molecular mass of about 2500 (A-5) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Poly[2-(2-ethoxyethoxyethyl)guanidinium hydroxide] having a mean molecular mass of about 3000 (A-6) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Polycondensate of poly(hexamethyleneguanidinium hydroxide) and poly[2-(2-ethoxy)ethoxyethyl)guanidinium hydroxide] in a molar ratio of 3:1 (A-7) and at least one further active compound selected from the group consisting of (B-3), (B-5), (B-12), (B-17), (B-18), (B-19), (B-30), (B-41), (B-46), (B-47), (B-51), (C-2), (C-3), (C-4), (C-6), (C-12), (C-14), (C-15), (C-16), (C-17), (C-19), (C-25), (C-41), (D-4), (D-6), (D-8), (D-9), (D-10), (D-11), (D-12), (D-13), (D-15), (E-2), (E-6), (E-8), (E-10), (E-11), (E-12), (E-13), (E-14), (F-4), (F-10), (F-12), (F-14), (F-15), (F-16), (F-17), (F-18), (F-20), (F-21), (F-22), (F-24), (F-26), (F-27), (F-28), (F-29), (G-1), (G-2), (G-7), (G-9), (G-10), (G-14), (G-15), (G-16), (G-18), (G-19), (G-20), (G-31), (G-33), (G-34), (G-35), (G-39), (G-46), (G-47), (G-52), (G-54), (G-55), (G-57), (G-59), (G-60), (G-61), (G-64), (G-73), (G-74), (G-86), (G-93), (G-101), (G-138), (G-146).

Very particular preference is given to the active compound combinations listed below and to compositions comprising these active compound combinations:

Poly[2-(2-ethoxyethoxyethyl)guanidinium hydroxide] having a mean molecular mass of about 1000 (A-1) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

Poly[2-(2-ethoxyethoxyethyl)guanidinium hydroxide] having a mean molecular mass of about 1500 (A-2) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

Poly[2-(2-ethoxyethoxyethyl)guanidinium hydroxide] having a mean molecular mass of about 500 (A-3) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

Poly[2-(2-ethoxyethoxyethyl)guanidinium hydroxide] having a mean molecular mass of about 2000 (A-4) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

Poly[2-(2-ethoxyethoxyethyl)guanidinium hydroxide] having a mean molecular mass of about 2500 (A-5) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

Poly[2-(2-ethoxyethoxyethyl)guanidinium hydroxide] having a mean molecular mass of about 3000 (A-6) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

Polycondensate poly(hexamethyleneguanidinium hydroxide) and poly[2-(2-ethoxy)ethoxyethyl)guanidinium hydroxide] in a molar ratio of 3:1 (A-7) and at least one further active compound selected from the group consisting of (B-3), (B-7), (B-12), (B-16), (B-26), (B-39), (B-41), (B-46), (B-47), (B-52), (C-2), (C-3), (C-4), (C-6), (C-12), (C-14), (C-41), (D-2) (D-4), (D-8), (D-13), (D-15), (E-6), (F-4), (F-20), (F-24), (G-10), (G-15), (G-16), (G-18), (G-31), (G-35), (G-55), (G-60), (G-64), (G-73), (G-86), (G-101).

If the active compounds in the active compound combinations according to the invention are present in certain weight ratios, the synergistic effect is particularly pronounced. However, the weight ratios of the active compounds in the active compound combinations can be varied within a relatively wide range.

In general, 0.01-100, preferably 0.02-50, particularly preferably 0.05-20, very particularly preferably 0.01-10 parts by weight of active compound selected from groups (B), (C), (D), (E), (F) and (G) are present per part by weight of guanidium hydroxide (component A). Moreover, it is possible to employ 0.01-95; 0.11-90; 0.012-85; 0.0125-80; 0.013-75; 0.014-70; 0.015-65; 0.017-60; 0.018-55; 0.022-45; 0.025-40; 0.03-35; 0.033-30; 0.04-25; 0.067-15; 0.2-5; 0.25-4; 0.33-3; 0.5-2 parts by weight of active compound selected from groups (B), (C), (D), (E), (F) and (G) per part by weight of guanidium hydroxide (component A).

According to the invention, the term “active compound combination” means various possible combinations of the three active compounds mentioned above, such as, for example, readymixes, tank mixes (which is to be understood as meaning spray mixtures prepared prior to application from the formulations of the individual active compounds by mixing and diluting) or combinations thereof (for example, a binary readymix of two of the active compounds mentioned above is converted with a formulation of the third individual substance into a tank mix). According to the invention, the individual active compounds may also be used successively, i.e. one after the other, within a reasonable interval of a few hours or days, and, in the treatment of seed, also, for example, by applying a plurality of layers comprising different active compounds. Preferably, the order in which the individual active compounds may be employed is immaterial.

The present invention furthermore relates to compositions comprising the active compound combinations according to the invention. Preferably, the compositions are fungicidal compositions comprising agriculturally suitable carriers or extenders.

According to the invention, carrier is to be understood as meaning a natural or synthetic, organic or inorganic substance which is mixed or combined with the active compounds for better applicability, in particular for application to plants or plant parts or seeds. The carrier, which may be solid or liquid, is generally inert and should be suitable for use in agriculture.

Suitable solid carriers are: for example ammonium salts and natural ground minerals, such as kaolins, clays, talc, chalk, quartz, attapulgite, montmorillonite or diatomaceous earth, and ground synthetic minerals, such as finely divided silica, alumina and natural or synthetic silicates, resins, waxes, solid fertilizers, water, alcohols, especially butanol, organic solvents, mineral oils and vegetable oils, and also derivatives thereof. It is also possible to use mixtures of such carriers. Solid carriers suitable for granules are: for example crushed and fractionated natural minerals, such as calcite, marble, pumice, sepiolite, dolomite, and also synthetic granules of inorganic and organic meals and also granules of organic material, such as sawdust, coconut shells, maize cobs and tobacco stalks. Suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, and also protein hydrolysates. Suitable dispersants are: for example lignosulphite waste liquors and methylcellulose.

Suitable liquefied gaseous extenders or carriers are liquids which are gaseous at ambient temperature and under atmospheric pressure, for example aerosol propellants, such as butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible for example, to use organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may comprise additional further components, such as, for example, surfactants. Suitable surfactants are emulsifiers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates. The presence of a surfactant is required if one of the active compounds and/or one of the inert carriers is insoluble in water and when the application takes place in water. The proportion of surfactants is between 5 and 40 percent by weight of the composition according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

If appropriate, other additional components may also be present, for example protective colloids, binders, adhesives, thickeners, thixotropic substances, penetrants, stabilizers, sequestering agents, complex formers. In general, the active compounds can be combined with any solid or liquid additive customarily used for formulation purposes.

In general, the compositions according to the invention comprise between 0.05 and 99 percent by weight of the active compound combination according to the invention, preferably between 10 and 70 percent by weight, particularly preferably between 20 and 50 percent by weight, most preferably 25 percent by weight.

The active compound combinations or compositions according to the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, microgranules, microgranules, oil-dispersible powders, oil-miscible flowable concentrates, oil-miscible liquids, foams, pastes, pesticide-coated seed, suspension concentrates, suspoemulsion concentrates, soluble concentrates, suspensions, wettable powders, soluble powders, dusts and granules, water-soluble granules or tablets, water-soluble powders for the treatment of seed, wettable powders, natural products and synthetic substances impregnated with active compound, and also microencapsulations in polymeric substances and in coating materials for seed, and also ULV cold-fogging and warm-fogging formulations.

The formulations mentioned can be prepared in a manner known per se, for example by mixing the active compounds or the active compound combinations with at least one additive. Suitable additives are all customary formulation auxiliaries, such as, for example, organic solvents, extenders, solvents or diluents, solid carriers and fillers, surfactants (such as adjuvants, emulsifiers, dispersants, protective colloids, wetting agents and tackifiers), dispersants and/or binders or fixatives, preservatives, dyes and pigments, defoamers, inorganic and organic thickeners, water repellents, if appropriate siccatives and UV stabilizers, gibberellins and also water and further processing auxiliaries. Depending on the formulation type to be prepared in each case, further processing steps such as, for example, wet grinding, dry grinding or granulation may be required.

Organic diluents that may be present are all polar and non-polar organic solvents that are customarily used for such purposes. Preferred are ketones, such as methyl isobutyl ketone and cyclohexanone, furthermore amides, such as dimethylformamide and alkanecarboxamides, such as N,N-dimethyldecanamide and N,N-dimethyloctanamide, furthermore cyclic compounds, such as N-methylpyrrolidone, N-octylpyrrolidone, N-dodecylpyrrolidone, N-octylcaprolactam, N-dodecylcaprolactam and butyrolactone, additionally strongly polar solvents, such as dimethyl sulphoxide, furthermore aromatic hydrocarbons, such as xylene, Solvesso™, mineral oils, such as white spirit, petroleum, alkylbenzenes and spindle oil, moreover esters, such as propylene glycol monomethyl ether acetate, dibutyl adipate, hexyl acetate, heptyl acetate, tri-n-butyl citrate and di-n-butyl phthalate, and furthermore alcohols, such as, for example, benzyl alcohol and 1-methoxy-2-propanol.

Solid carriers suitable for granules are: for example crushed and fractionated natural minerals, such as calcite, marble, pumice, sepiolite, dolomite, and also synthetic granules of inorganic and organic meals and also granules of organic material, such as sawdust, coconut shells, maize cobs and tobacco stalks.

Suitable surfactants (adjuvants, emulsifiers, dispersants, protective colloids, wetting agents and tackifiers) are customary ionic and nonionic substances. Examples which may be mentioned are ethoxylated nonylphenols, polyalkylene glycol ethers of straight-chain or branched alcohols, products of reactions of alkylphenols with ethylene oxide and/or propylene oxide, products of reactions of fatty amines with ethylene oxide and/or propylene oxide, furthermore fatty esters, alkylsulphonates, alkyl sulphates, alkyl ether sulphates, alkyl ether phosphates, aryl sulphates, ethoxylated arylalkylphenols, such as, for example, tristyrylphenol ethoxylates, furthermore ethoxylated and propoxylated arylalkylphenols and also sulphated or phosphated arylalkylphenol ethoxylates or ethoxy- and propoxylates. Mention may furthermore be made of natural and synthetic water-soluble polymers, such as lignosulphonates, gelatine, gum arabic, phospholipids, starch, hydrophobically modified starch and cellulose derivatives, in particular cellulose esters and cellulose ethers, furthermore polyvinyl alcohol, polyvinyl acetate, polyvinylpyrrolidone, polyacrylic acid, polymethacrylic acid and copolymers of (meth)acrylic acid and (meth)acrylic acid esters, and moreover also alkali metal hydroxide-neutralized copolymers of methacrylic acid and methacrylic ester and condensates of optionally substituted naphthalenesulphonic acid salts with formaldehyde.

Suitable solid fillers and carriers are all substances customarily used for this purpose in crop pretection compositions. Inorganic particles, such as carbonates, silicates, sulphates and oxides having a mean particle size of from 0.005 to 20 μm, particularly preferably from 0.02 to 10 μm, may be mentioned as being preferred. Examples which may be mentioned are ammonium sulphate, ammonium phosphate, urea, calcium carbonate, calcium sulphate, magnesium sulphate, magnesium oxide, aluminium oxide, silicon dioxide, finely divided silicic acid, silica gels, natural and synthetic silicates and alumosilicates and vegetable products such as cereal meal, wood powder and cellulose powder.

Suitable colorants that may be present in the seed dressing formulations to be used according to the invention include all colorants customary for such purposes. Use may be made both of pigments, of sparing solubility in water, and of dyes, which are soluble in water. Examples that may be mentioned include the colorants known under the designations Rhodamin B, C.I. Pigment Red 112 and C.I. Solvent Red 1. The colorants used can be inorganic pigments, for example iron oxide, titanium oxide, Prussian Blue, and organic dyes, such as alizarin, azo and metal phthalocyanine dyes, and trace nutrients, such as salts of iron, manganese, boron, copper, cobalt, molybdenum and zinc.

Suitable wetting agents that may be present in the seed dressing formulations to be used according to the invention include all substances which promote wetting and are customary in the formulation of agrochemically active compounds. Preference is given to using alkylnaphthalenesulphonates, such as diisopropyl- or diisobutylnaphthalenesulphonates.

Suitable dispersants and/or emulsifiers that may be present in the seed dressing formulations to be used according to the invention include all nonionic, anionic and cationic dispersants which are customary in the formulation of agrochemically active compounds. Preference is given to using nonionic or anionic dispersants or mixtures of nonionic or anionic dispersants. Particularly suitable nonionic dispersants are ethylene oxide/propylene oxide block polymers, alkylphenol polyglycol ethers, and also tristryrylphenol polyglycol ethers and their phosphated or sulphated derivatives. Particularly suitable anionic dispersants are lignosulphonates, polyacrylic acid salts and arylsulphonate/formaldehyde condensates.

Defoamers that may be present in the seed dressing formulations to be used according to the invention include all foam-inhibiting compounds which are customary in the formulation of agrochemically active compounds. Preference is given to using silicone defoamers, magnesium stearate, silicone emulsions, long-chain alcohols, fatty acids and their salts and also organofluorine compounds and mixtures thereof.

Preservatives that may be present in the seed dressing formulations to be used according to the invention include all compounds which can be used for such purposes in agrochemical compositions. By way of example, mention may be made of dichlorophen and benzyl alcohol hemiformal.

Secondary thickeners that may be present in the seed dressing formulations to be used according to the invention include all compounds which can be used for such purposes in agrochemical compositions. Preference is given to cellulose derivatives, acrylic acid derivatives, polysaccharides, such as xanthan gum or Veegum, modified clays, phyllosilicates, such as attapulgite and bentonite, and also finely divided silicic acids.

Suitable adhesives that may be present in the seed dressing formulations to be used according to the invention include all customary binders which can be used in seed dressings. Polyvinylpyrrolidone, polyvinyl acetate, polyvinyl alcohol and tylose may be mentioned as being preferred.

Suitable gibberellins that may be present in the seed dressing formulations to be used according to the invention are preferably the gibberellins A1, A3 (=gibberellic acid), A4 and A7; particular preference is given to using gibberellic acid. The gibberellins are known (cf. R. Wegler “Chemie der Pflanzen-schutz- and Schädlingsbekämpfungsmittel” [Chemistry of Crop Protection Agents and Pesticides], Vol. 2, Springer Verlag, 1970, pp. 401-412).

The formulations generally comprise between 0.1 and 95% by weight of active compound, preferably between 0.5 and 90%.

The active compound combinations according to the invention can be present in commercial formulations and in the use forms prepared from these formulations as a mixture with other active compounds, such as insecticides, attractants, sterilants, bactericides, acaricides, nematicides, fungicides, growth regulators or herbicides. A mixture with fertilizers is also possible.

The treatment according to the invention of the plants and plant parts with the active compound combinations or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by incrusting, by coating with one or more coats, etc. Preference is given to application by dipping, spraying, atomizing, irrigating, evaporating, dusting, fogging, broadcasting, foaming, painting, spreading-on, watering (drenching) and drip irrigating.

The application of the formulations is carried out in accordance with customary agricultural practice in a manner adapted to the application forms. Customary applications are, for example, dilution with water and spraying of the resulting spray liquor, application after dilution with oil, direct application without dilution, seed dressing or soil application of carrier granules.

The active compound content of the application forms prepared from the commercial formulations can vary within wide limits. The active compound concentration of the application forms can be from 0.0000001 up to 95% by weight of active compound, preferably between 0.0001 and 2% by weight.

The compositions according to the invention do not only comprise ready-to-use compositions which can be applied with suitable apparatus to the plant or the seed, but also commercial concentrates which have to be diluted with water prior to use.

The active compound combinations or compositions according to the invention have strong microbicidal activity and can be used for controlling unwanted microorganisms, such as fungi and bacteria, in crop protection.

In crop protection, fungicides can be used for controlling Plasmodiophoromycetes, Oomycetes, Chytridiomycetes, Zygomycetes, Ascomycetes, Basidiomycetes and Deuteromycetes.

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compound combinations or compositions according to the invention, which are applied to the seed, the plant or plant parts, the fruit or the soil in which the plants grow. Preference is given to application onto the plant or the plant parts, the fruits or the soil in which the plants grow.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds. Preference is given to the treatment of the plants and the above-ground and below-ground parts and organs of the plants, such as shoot, leaf, flower and root, examples which may be mentioned being leaves, needles, stems, trunks, flowers, fruits.

The following plants may be mentioned as plants which can be treated according to the invention: cotton, flax, grapevines, fruit, vegetable, such as Rosaceae sp. (for example pomaceous fruit, such as apples and pears, but also stone fruit, such as apricots, cherries, almonds and peaches and soft fruit such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onions), Papilionaceae sp. (for example peas); major crop plants, such Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflowers, brussel sprouts, pak choi, kohlrabi, garden radish, and also oilseed rape, mustard, horseradish and cress), Fabacae sp. (for example beans, peas), Papilionaceae sp. (for example soya beans), Solanaceae sp. (for example potatoes), Chenopodiaceae sp. (for example sugarbeet, fodderbeet, swiss chard, beetroot); crop plants and ornamental plants in garden and forest; and also in each case genetically modified varieties of these plants. Preferably, cereal plants are treated according to the invention.

The method according to the invention for controlling phytopathogenic fungi can also be employed for treating genetically modified organisms, for example plants or seeds. Genetically modified plants are plants whose genome has, stably integrated, a certain heterologous gene coding for a certain protein. Here, “heterologous gene” is meant to be understood as a gene which confers novel agronomical properties on the transformed plant, or a gene which improves the agronomical quality of the modified plant.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The terms “parts”, “parts of plants” and “plant parts” have been explained above. Particularly preferably, plants of the plant cultivars which are in each case commercially available or in use are treated according to the invention.

Depending on the plant species or plant cultivars, their location and growth conditions (soil, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defense of the plants against animal and microBial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape, and also fruit plants (with the fruits apples, pears, citrus fruits and grapes), and particular emphasis is given to maize, soya beans, potatoes, cotton, and oilseed rape. “Traits” that are emphasized are in particular increased defense of the plants against insects by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potatoes). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The method according to the invention for controlling unwanted fungi can also be employed for protecting storage goods. Here, storage goods are to be understood as meaning natural substances of vegetable or animal origin or processed products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compound combinations according to the invention can prevent disadvantageous effects, such as rotting, decay, discoloration, decoloration or the formation of mould.

A further application of the active compound combinations and compositions according to the invention is the protection of wood and timber. The insecticidal and fungicidal compositions or concentrates used for protecting wood and timber comprise the active compound according to the invention in a concentration of from 0.0001 to 95% by weight, in particular from 0.001 to 60% by weight.

The active compound combinations and compositions according to the invention can likewise be employed for protecting against colonization of objects, in particular ship hulls, sieves, nets, buildings, quays and signalling installations, which are in contact with sea water or brackish water.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collo-cygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;

Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum; Verticilium species, such as, for example, Verticilium alboatrum;

Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cancerous diseases, galls and witches' broom caused, for example, by Nectria species, such as, for example, Nectria galligena;

Wilt diseases caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myri otylum, Pythium ultimum), rhizoctonia root rot, stem decay, and damping-off (Rhizoctonia solani), sclerotinia stem decay (Sclerotinia sclerotiorum), sclerotinia Southern blight (Sclerotinia rolfsii), thielaviopsis root rot (Thielaviopsis basicola).

The following fungal and bacterial plant diseases are controlled with particular preference: early blight (Alternaria solani), grey mould (Botryis cinerea), late blight (Phytophthora infestans), downy mildew (Plasmopara viticola), apple scab (Venturia inaequalis), fusarioses (caused, for example, by Fusarium culmorum and Fusarium graminearum), leaf blotch (Septoria tritici), snow mould (Microdochium nivale) and fire blight (Erwinia amylovora).

It is also possible to control resistant strains of the organisms mentioned above.

The application rate of the active compound combinations according to the invention is
when treating leaves: from 0.1 to 10 000 g/ha, preferably from 10 to 1000 g/ha, particularly preferably from 50 to 300 g/ha (when the application is carried out by watering or dripping, it may even be possible to reduce the application rate, in particular when inert substrates such as rock wool or perlite are used);
when treating seed: from 2 to 200 g per 100 kg of seed, preferably from 3 to 150 g pro 100 kg of seed, particularly preferably from 2.5 to 25 g per 100 kg of seed, very particularly preferably from 2.5 to 12.5 g per 100 kg of seed;
when treating the soil: from 0.1 to 10 000 g/ha, preferably from 1 to 5000 g/ha.

These application rates are mentioned only by way of example and not by way of limitation in the sense of the invention.

The active compound combinations or compositions according to the invention can thus be employed for protecting plants for a certain period of time after treatment against attack by the pathogens mentioned. The period for which protection is provided extends generally for 1 to 28 days, preferably 1 to 14 days, after the treatment of the plants with the active compounds, or up to 200 days after a seed treatment.

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec. inter alia.

The invention furthermore comprises a method for treating seed where the individual active compounds are applied simultaneously to the seed. Moreover, the invention comprises a method for treating seed where the individual active compounds are applied successively to the seed. Moreover, the invention comprises a method for treating seed where an individual active compound is applied first, followed by a binary mixture of the two other active compounds. Alternatively, it is also possible to apply to the seed first a binary mixture, followed by the remaining individual active compound. If active compounds and/or individual active compounds and binary mixtures are applied separately, this is preferably carried out in different layers. These layers may additionally be separated by layers without active compound.

The invention furthermore relates to seed treated according to one of the methods described in the preceding paragraph.

The active compound combinations or compositions according to the invention are especially suitable for treating seed. A large part of the damage to crop plants caused by harmful organisms is triggered by an infection of the seed during storage or after sowing as well as during and after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

The control of phytopathogenic fungi by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with the additional application of crop protection agents after sowing or after the emergence of the plants or which at least considerably reduce additional application. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

Accordingly, the present invention also relates in particular to a method for protecting seed and germinating plants against attack by phytopathogenic fungi by treating the seed with a composition according to the invention. The invention also relates to the use of the compositions according to the invention for treating seed for protecting the seed and the germinating plant against phytopathogenic fungi. Furthermore, the invention relates to seed treated with a composition according to the invention for protection against phytopathogenic fungi.

The control of phytopathogenic fungi which damage plants post-emergence is carried out primarily by treating the soil and the above-ground parts of plants with crop protection compositions. Owing to the concerns regarding a possible impact of the crop protection composition on the environment and the health of humans and animals, there are efforts to reduce the amount of active compounds applied.

One of the advantages of the present invention is that, because of the particular systemic properties of the compositions according to the invention, treatment of the seed with these compositions not only protects the seed itself, but also the resulting plants after emergence, from phytopathogenic fungi. In this manner, the immediate treatment of the crop at the time of sowing or shortly thereafter can be dispensed with.

It is also considered to be advantageous that the mixtures according to the invention can be used in particular also for transgenic seed where the plant growing from this seed is capable of expressing a protein which acts against pests. By treating such seed with the active compound combinations or compositions according to the invention, even by the expression of the, for example, insecticidal protein, certain pests may be controlled. Surprisingly, a further synergistic effect may be observed here, which additionally increases the effectiveness of the protection against attack by pests.

The compositions according to the invention are suitable for protecting seed of any plant variety employed in agriculture, in the greenhouse, in forests or in horticulture or viticulture. In particular, this takes the form of seed of maize, peanuts, oilseed rape, poppies, olives, coconuts, cocao, soya bean, beets (for example sugar beets and fodder beets), rice, millet, wheat, barley, rye, triticale, oats, cotton, potatoes, sunflowers, sugar cane, tobacco, beans, coffee, vegetables (such as tomatoes, cucumbers, onions and lettuce), lawn and ornamental plants (also see above).

As already described, the treatment of transgenic seed with the active compound combinations or compositions according to the invention is of particular importance. This refers to the seed of plants containing at least one heterologous gene which allows the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. Preferably, this heterologous gene is from Bacillus sp., the gene product having activity against the European corn borer and/or the Western corn rootworm. Particularly preferably, the heterologous gene originates from Bacillus thuringiensis.

In the context of the present invention, the active compound combinations or compositions according to the invention are applied on their own or in a suitable formulation to the seed. Preferably, the seed is treated in a state in which it is sufficiently stable so that the treatment does not cause any damage. In general, treatment of the seed may take place at any point in time between harvesting and sowing. Usually, the seed used is separated from the plant and freed from cobs, shells, stalks, coats, hairs or the flesh of the fruits. Thus, it is possible to use, for example, seed which has been harvested, cleaned and dried to a moisture content of less than 15% by weight. Alternatively, it is also possible to use seed which, after drying, has been treated, for example, with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the compositions to the seed in the form of a suitable formulation. Suitable formulations and methods for the treatment of seed are known to the person skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

According to a further aspect of the present invention, in the active compound combinations or compositions according to the invention the ratio of the three components is preferably chosen such that a synergistic effect is obtained. Here, a “synergistic effect” is to be understood, for example, as described by Colby in the article “Calculation of the synergistic and antagonistic responses of herbicide combinations” (in Weeds, 1967, 15, 20-22).

According to Colby, a synergistic (superadditive) effect is present when the actual fungicidal activity is greater than calculated. In this case, the efficacy actually observed has to be greater than the value for the expected efficacy (E) calculated using the formula given below.

If

X is the efficacy when active compound A is applied at an application rate of m g/ha,
Y is the efficacy when active compound B is applied at an application rate of n g/ha,
Z is the efficacy when active compound C is applied at an application rate of r g/ha,
E₁ is the efficacy when the active compounds A and B are applied at application rates of m and n g/ha, respectively, and
E₂ is the efficacy when the active compounds A and B and C are applied at application rates of m and n and r g/ha, respectively,
then for a combination of 2 active compounds:

$${E}_{1} = X + Y - \frac{X \cdot Y}{100}$$

and for a combination of 3 active compounds:

$${E}_{2} = X + Y + Z - ( \frac{X \cdot Y + X \cdot Z + Y \cdot Z}{100} ) + \frac{X \cdot Y \cdot Z}{10000}$$

Here, the efficacy is determined in %. 0% means an efficacy which corresponds to that of the control, whereas an efficacy of 100% means that no infection is observed.

A further way of demonstrating a synergistic effect is the method of Tammes (cf. “Isoboles, a graphic representation of synergism in pesticides” in Neth. J. Plant Path., 1964, 70, 73-80).

The invention is illustrated by the examples below. However, the invention is not limited to the examples.

### USE EXAMPLES

### Example A

### Alternaria Test (Tomato)/Protective

To test for protective activity, young tomato plants are sprayed with the active compound preparation in an aqueous solution at the stated application rate. 1 day after the treatment, the plants are inoculated with a spore suspension of Alternaria solani and then remain at a relative humidity of 100% and 20° C. for 24 h. The plants then remain at a relative atmospheric humidity of 96% and a temperature of 20° C. Evaluation is carried out 7 days after inoculation. Here, 0% means an efficacy which corresponds to that of the control, whereas an efficacy of 100% means that no infection is observed.

TABLE A
Alternaria test (tomato)/protective

| Active | Active compound | Efficacy |
|---|---|---|
| compound | application rate in ppm | in % |
| (A-7) | 2000 | 95 |

### Example B

### Botrytis Test (Cucumber)/Protective

To test for protective activity, young cucumber plants are sprayed with the active compound preparation in an aqueous solution at the stated application rate. 1 day after the treatment, the plants are inoculated with a spore suspension of Botryis cinerea and then remain at a relative humidity of 100% and 22° C. for 48 h. The plants then remain at a relative atmospheric humidity of 96% and a temperature of 14° C. Evaluation is carried out 5-6 days after inoculation. Here, 0% means an efficacy which corresponds to that of the control, whereas an efficacy of 100% means that no infection is observed.

TABLE B
Botrytis test (cucumber)/protective

| Active | Active compound | Efficacy |
|---|---|---|
| compound | application rate in ppm | in % |
| (A-7) | 2000 | 100 |

### Example C

### Phytophthora Test (Tomato)/Protective

To test for protective activity, young tomato plants are sprayed with the active compound preparation in an aqueous solution at the stated application rate. 1 day after the treatment, the plants are inoculated with a spore suspension of Phytophthora infestans and then remain at a relative humidity of 100% and 20° C. for 24 h. The plants are then placed into a climatized cell at about 96% relative atmospheric humidity and a temperature of about 20° C. Evaluation is carried out 7 days after inoculation. Here, 0% means an efficacy which corresponds to that of the control, whereas an efficacy of 100% means that no infection is observed.

TABLE C
Phytophthora test (tomato)/protective

| Active | Active compound | Efficacy |
|---|---|---|
| compound | application rate in ppm | in % |
| (A-7) | 2000 | 90 |

### Example D

### Plasmopara Test (Grapevine)/Protective

To test for protective activity, young plants are sprayed with the active compound preparation in an aqueous solution at the stated application rate. After the spray coating has dried on, the plants are inoculated with an aqueous spore suspension of Plasmopara viticola inokuliert and then remain in an incubation cabin at about 20° C. and 100% relative atmospheric humidity for 1 day. The plants are then placed in a greenhouse at about 21° C. and about 90% atmospheric humidity for 4 days. The plants are then moistened and placed in an incubation cabin for 1 day. Evaluation is carried out 6 days after inovulation. Here, 0% means an efficacy which corresponds to that of the control, whereas an efficacy of 100% means that no infection is observed.

TABLE D
Plasmopara test (grapevine)/protective

| Active | Active compound | Efficacy |
|---|---|---|
| compound | application rate in ppm | in % |
| (A-7) | 2000 | 95 |

### Example E

### Venturia Test (Apple)/Protective

To test for protective activity, young plants are sprayed with the active compound preparation in an aqueous solution at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day. The plants are then placed in a greenhouse at about 21° C. and a relative atmospheric humidity of about 90%. Evaluation is carried out 10 days after inoculation. Here, 0% means an efficacy which corresponds to that of the control, whereas an efficacy of 100% means that no infection is observed.

TABLE E
Venturia test (apple)/protective

| Active | Active compound | Efficacy |
|---|---|---|
| compound | application rate in ppm | in % |
| (A-7) | 2000 | 98 |

### Example F

### Fusarium culmorum Test (Wheat)/Protective

To test for protective activity, young plants are sprayed with the active compound preparation in an aqueous solution at the stated application rate. After the spray coating has dried on, the plants are inoculated with an aqueous conidia suspension of Fusarium culmorum. The plants are then placed in a greenhouse under translucent incubation hoods at about 22° C. and a relative atmospheric humidity of about 100%. Evaluation is carried out 6 days after inoculation. Here, 0% means an efficacy which corresponds to that of the control, whereas an efficacy of 100% means that no infection is observed.

TABLE F
Fusarium culmorum test (wheat)/protective

| | Active compound application rate in | |
|---|---|---|
| Active compound | ppm | Efficacy in % |
| (A-7) | 2000 | 80 |

### Example G

### Fusarium graminearum Test (Barley)/Protective

To test for protective activity, young plants are sprayed with the active compound preparation in an aqueous solution at the stated application rate. After the spray coating has dried on, the plants are inoculated with an aqueous conidia suspension of Fusarium graminearum. The plants are then placed in a greenhouse under translucent incubation hoods at about 22° C. and a relative atmospheric humidity of about 100%. Evaluation is carried out 5 days after inoculation. Here, 0% means an efficacy which corresponds to that of the control, whereas an efficacy of 100% means that no infection is observed.

TABLE G
Fusarium graminearum test (barley)/protective

| | Active compound application rate in | |
|---|---|---|
| Active compound | ppm | Efficacy in % |
| (A-7) | 2000 | 86 |

### Example H

### Septoria tritici Test (Wheat)/Protective

To test for protective activity, young plants are sprayed with the active compound preparation in an aqueous solution at the stated application rate. After the spray coating has dried on, the plants are inoculated with an aqueous spore suspension of Septoria tritici. The plants remain in an incubation cabin at about 20° C. and a relative atmospheric humidity of about 100% for 48 hours. The plants are then placed under a translucent incubation hood at about 15° C. and a relative atmospheric humidity of about 100% for 60 hours. The plants then remain in a greenhouse at a temperature of about 15° C. and a relative atmospheric humidity of about 80%. Evaluation is carried out 21 days after inoculation. Here, 0% means an efficacy which corresponds to that of the control, whereas an efficacy of 100% means that no infection is observed.

TABLE H
Septoria tritici test (wheat)/protective

| | Active compound application rate in | |
|---|---|---|
| Active compound | ppm | Efficacy in % |
| (A-7) | 2000 | 75 |

### Example I

### Microdochium nivale Test (Lawn)/Protective

Lawn seed (100% Agrostis stolonifera) was sown in dishes of the dimensions 38 cm×15 cm×9 cm. 7 days after sowing, the lawn was cut prior to preventive guanidinium hydroxide application at application rates of 2000, 1000, 500 and 100 g/ha. The water application rates were 1000 l/ha (converted). 24 hours after the preventive application of the active compound, the lawn was inoculated by placing of wheat grains infected with Microdochium nivale. In each case 3 wheat grains were placed into a single dish. Scoring of the subsequent fungal growth in the lawn dishes was carried out after 5, 7, 14 and 21 days. In the dishes which had been treated with guanidinium hydroxide, an inhibition of the growth of Microdochium nivale of up to 30% (Abbott) was observed.

TABLE I
Microdochium nivale test (lawn)/protective

| | | Efficacy |
|---|---|---|
| Test component | Application rate in ppm | [in % Abbott] |
| (A-7) | 2000 | 30 |
| | 1000 | 27 |
| | 500 | 9 |
| | 100 | 0 |

### Example J

### Phytophthora Test (Tomato)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, the plants are inoculated with an aqueous spore suspension of Phytophthora infestans. The plants are then placed in an incubation cabin at about 20° C. and 100% relative atmospheric humidity. Evaluation is carried out 3 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE J-1
Phytophthora test (tomato)/protective

| | Active | | |
|---|---|---|---|
| | compound | | |
| Active compound/ | application | Efficacy in % | |
| active compound combination | rate in ppm | found* | calc.** |
| (A-7) | 200 | 41 | |
| | 50 | 13 | |
| (G-15) benthiavalicarb | 0.5 | 23 | |
| (E-6) fluopicolide | 2 | 9 | |
| (G-18) iprovalicarb | 2 | 30 | |
| (G-55) metalaxyl-M | 2.5 | 10 | |
| (A-7) + (G-15) benthiavalicarb 1:0.01 | 50 + 0.5 | 52 | 33 |
| (A-7) + (E-6) fluopicolide 1:0.01 | 200 + 2 | 68 | 46 |
| (A-7) + (G-18) iprovalicarb 1:0.01 | 200 + 2 | 75 | 59 |
| (A-7) + (G-55) metalaxyl-M 1:0.05 | 50 + 2.5 | 46 | 22 |

*found = activity found
**calc. = activity calculated according to the Colby formula

TABLE J-2
Phytophthora test (tomato)/protective

| | Active compound | | |
|---|---|---|---|
| Active compound/ | application | Efficacy in % | |
| active compound combination | rate in ppm | found* | calc.** |
| (A-7) | 200 | 41 | |
| | 50 | 13 | |
| (F-20) mancozeb | 25 | 20 | |
| (G-35) propamocarb hydrochloride | 100 | 0 | |
| (F-24) propineb | 100 | 15 | |
| (A-7) + (F-20) mancozeb 1:0.5 | 50 + 25 | 65 | 53 |
| (A-7) + (G-35) propamocarb | 50 + 100 | 56 | 41 |
| hydrochloride 1:2 | | | |
| (A-7) + (F-24) propineb 1:0.5 | 200 + 100 | 66 | 54 |

*found = activity found
**calc. = activity calculated according to the Colby formula

### Example K

### Plasmopara Test (Grapevine)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, the plants are inoculated with an aqueous spore suspension of Plasmopara viticola and then remain in an incubation cabin at about 20° C. and 100% relative atmospheric humidity for 1 day. The plants are then placed in a greenhouse at about 21° C. and about 90% atmospheric humidity for 4 days. The plants are then moistened and placed in an incubation cabin for 1 day. Evaluation is carried out 6 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE K
Plasmopara test (grapevine)/protective

| | Active | | |
|---|---|---|---|
| | compound | | |
| Active compound/ | application | Efficacy in % | |
| active compound combination | rate in ppm | found* | calc.** |
| (A-7) | 200 | 51 | |
| | 100 | 46 | |
| | 50 | 25 | |
| (G-73) cymoxanil | 50 | 0 | |
| (G-16) dimethomorph | 1 | 0 | |
| (D-2) fenamidone | 1 | 19 | |
| (G-64) fluazinam | 5 | 0 | |
| (G-86) fosetyl-Al | 200 | 0 | |
| (G-18) iprovalicarb | 0.5 | 0 | |
| (F-20) mancozeb | 100 | 5 | |
| (G-101) phosphorous acid | 400 | 30 | |
| (G-35) propamocarb hydrochloride | 400 | 0 | |
| (F-24) propineb | 100 | 51 | |
| (A-7) + (G-73) cymoxanil 1:0.5 | 100 + 50 | 65 | 46 |
| (A-7) + (G-16) dimethomorph 1:0.01 | 100 + 1 | 62 | 46 |
| (A-7) + (D-2) fenamidone 1:0.01 | 100 + 1 | 73 | 56 |
| (A-7) + (G-64) fluazinam 1:0.05 | 100 + 5 | 70 | 46 |
| (A-7) + (G-86) fosetyl-Al 1:2 | 100 + 200 | 78 | 46 |
| (A-7) + (G-18) iprovalicarb 1:0.01 | 50 + 0.5 | 45 | 25 |
| (A-7) + (F-20) mancozeb 1:0.5 | 200 + 100 | 66 | 53 |
| (A-7) + (G-101) phosphorous acid 1:2 | 200 + 400 | 85 | 66 |
| (A-7) + (G-35) propamocarb | 200 + 400 | 85 | 51 |
| hydrochloride 1:2 | | | |
| (A-7) + (F-24) propineb 1:0.5 | 200 + 100 | 89 | 76 |

*found = activity found
**calc. = activity calculated according to the Colby formula

### Example L

### Venturia Test (Apple)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day. The plants are then placed in a greenhouse at about 21° C. and a relative atmospheric humidity of about 90%. Evaluation is carried out 10 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE L
Venturia test (apple)/protective

| | Active | | |
|---|---|---|---|
| | compound | | |
| Active compound/ | application | Efficacy in % | |
| active compound combination | rate in ppm | found* | calc.** |
| (A-7) | 100 | 64 | |
| | 50 | 39 | |
| (B-3) bitertanol | 2.5 | 63 | |
| (C-3) boscalid | 5 | 36 | |
| (C-19) 3-(difluoromethyl)-1-methyl-N- | 5 | 35 | |
| [2-(1,1,2,2-tetrafluoroethoxy)phenyl]- | | | |
| 1H-pyrazole-4-carboxamide | | | |
| (C-12) penthiopyrad | 5 | 60 | |
| (A-7) + (B-3) bitertanol 1:0.5 | 50 + 2.5 | 95 | 77 |
| (A-7) + (C-3) boscalid 1:0.05 | 100 + 5 | 88 | 77 |
| (A-7) + (C-19) 3-(difluoromethyl)-1- | 100 + 5 | 91 | 77 |
| methyl-N-[2- (1,1,2,2- | | | |
| tetrafluoroethoxy)phenyl]- | | | |
| 1H-pyrazole-4-carboxamide 1:0.05 | | | |
| (A-7) + (C-12) penthiopyrad 1:0.05 | 100 + 5 | 98 | 86 |

*found = activity found
**calc. = activity calculated according to the Colby formula

### Example M

### Alternaria Test (Tomato)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, the plants are inoculated with an aqueous spore suspension of Alternaria solani. The plants are then placed in an incubation cabin at about 20° C. and 100% relative atmospheric humidity. Evaluation is carried out 3 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE M-1
Alternaria test (tomato)/protective

| | Active | | |
|---|---|---|---|
| | compound | | |
| Active compound/ | application | Efficacy in % | |
| active compound combination | rate in ppm | found* | calc.** |
| (A-7) | 250 | 17 | |
| | 125 | 0 | |
| (C-2) bixafen | 1.25 | 49 | |
| (C-6) fluopyram | 2.5 | 23 | |
| (D-8) fluoxastrobin | 5 | 35 | |
| (B-41) prothioconazole | 5 | 1 | |
| (C-14) N-[2-(1,3- | 2.5 | 0 | |
| dimethylbutyl)phenyl]- | | | |
| 5-fluoro-1,3-dimethyl-1H- | | | |
| pyrazole-4-carboxamide | | | |
| (B-47) tebuconazole | 5 | 29 | |
| (D-15) trifloxystrobin | 5 | 40 | |
| (A-7) + (C-2) bixafen 1:0.01 | 125 + 1.25 | 71 | 49 |
| (A-7) + (C-6) fluopyram 1:0.01 | 250 + 2.5 | 50 | 36 |
| (A-7) + (D-8) fluoxastrobin 1:0.02 | 250 + 5 | 80 | 46 |
| (A-7) + (B-41) | 250 + 5 | 50 | 18 |
| prothioconazole 1:0.02 | | | |
| (A-7) + (C-14) N-[2-(1,3- | 250 + 2.5 | 53 | 17 |
| dimethylbutyl)phenyl]-5- | | | |
| fluoro-1,3-dimethyl-1H-pyrazole- | | | |
| 4-carboxamide 1:0.01 | | | |
| (A-7) + (B-47) tebuconazole 1:0.02 | 250 + 5 | 53 | 41 |
| (A-7) + (D-15) trifloxystrobin 1:0.02 | 250 + 5 | 66 | 50 |

*found = activity found
**calc. = activity calculated according to the Colby formula

TABLE M-2
Alternaria test (tomato)/protective

| | Active compound | | |
|---|---|---|---|
| Active compound/ | application rate in | Efficacy in % | |
| active compound combination | ppm | found* | calc.** |
| (A-7) | 100 | 19 | |
| (F-20) mancozeb | 50 | 31 | |
| (D-13) pyraclostrobin | 5 | 49 | |
| (A-7) + (F-20) mancozeb 1:0.5 | 100 + 50 | 62 | 44 |
| (A-7) + (D-13) pyraclostrobin | 100 + 5 | 79 | 59 |
| 1:0.05 | | | |

*found = activity found
**calc. = activity calculated according to the Colby formula

### Example N

### Botrytis Test (Bean)/Protective

| | |
|---|---|
| Solvents: | 24.5 parts by weight of acetone |
| | 24.5 parts by weight of |
| | dimethylacetamide |
| Emulsifier: | 1 part by weight of alkylaryl |
| | polyglycol ether |

To produce a suitable preparation of active compound, 1 part by weight of active compound is mixed with the stated amounts of solvents and emulsifier, and the concentrate is diluted with water to the desired concentration. To test for protective activity, young plants are sprayed with the active compound preparation at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity. 2 days after the inoculation, the size of the infected areas on the leaves is evaluated. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE N
Botrytis test (bean)/protective

| | | Active compound | Efficacy in % | |
|---|---|---|---|---|
| Active compound/active compound combination | | application rate in ppm | found* | calc.** |
| (A-7) | | 50 | 15 | |
| (B-16) fenhexamid | | 2.5 | 0 | |
| (G-31) iprodione | | 12.5 | 11 | |
| (G-10) pyrimethanil | | 12.5 | 13 | |
| (A-7) + (B-16) fenhexamid | 1:0.05 | 50 + 2.5 | 45 | 15 |
| (A-7) + (G-31) iprodione | 1:0.25 | 50 + 12.5 | 48 | 24 |
| (A-7) + (G-10) pyrimethanil | 1:0.25 | 50 + 12.5 | 50 | 26 |

*found = activity found
**calc. = activity calculated according to the Colby formula

### Example O

### Blumeria graminis Test (Barley)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are dusted with spores of Blumeria graminis f. sp. hordei. The plants are placed in a greenhouse at a temperature of about 18° C. and a relative atmospheric humidity of about 80% to promote the development of mildew pustules. Evaluation is carried out 7 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE O
Blumeria graminis test (barley)/protective

| | | Active compound | Efficacy in % | |
|---|---|---|---|---|
| Active compound/active compound combination | | application rate in ppm | found* | calc.** |
| (A-7) | | 250 | 0 | |
| (C-19) 3-(difluoromethyl)-1-methyl-N-[2-(1,1,2,2- | | 125 | 78 | |
| tetrafluoroethoxy)phenyl]-1H-pyrazole-4- | | | | |
| carboxamide | | | | |
| (A-7) + (C-19) 3-(difluoromethyl)-1-methyl-N-[2- | 2:1 | 250 + 125 | 89 | 78 |

(1,1,2,2-tetrafluroethoxy)phenyl]-1H-pyrazole-4-
carboxamide

*found = activity found
**calc. = activity calculated according to the Colby formula

### Example P

### Blumeria graminis Test (Barley)/Curative

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for curative activity, young plants are dusted with spores of Blumeria graminis f. sp. hordei. 48 hours after the inoculation, the plants are sprayed with the active compound preparation at the stated application rate. The plants are placed in a greenhouse at a temperature of about 18° C. and a relative atmospheric humidity of about 80% to promote the development of mildew pustules. Evaluation is carried out 7 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE P
Blumeria graminis test (barley)/curative

| | | Active compound | Efficacy in % | |
|---|---|---|---|---|
| Active compound/active compound combination | | application rate in ppm | found* | calc.** |
| (A-7) | | 1000 | 0 | |
| | | 500 | 0 | |
| | | 250 | 0 | |
| (B-3) bitertanol | | 500 | 63 | |
| (C-2) bixafen | | 500 | 63 | |
| (B-52) tridemorph | | 250 | 75 | |
| (C-12) penthiopyrad | | 250 | 88 | |
| (D-8) fluoxastrobin | | 500 | 78 | |
| (C-14) N-[2-(1,3-dimethylbutyl)phenyl]-5-fluoro- | | 250 | 67 | |
| 1,3-dimethyl-1H-pyrazole-4-carboxamide | | | | |
| (B-7) difenoconazole | | 250 | 78 | |
| (C-41) isopyrazam | | 500 | 78 | |
| (A-7) + (B-3) bitertanol | 1:1 | 500 + 500 | 75 | 63 |
| (A-7) + (C-2) bixafen | 2:1 | 1000 + 500 | 88 | 63 |
| (A-7) + (B-52) tridemorph | 1:1 | 250 + 250 | 94 | 75 |
| (A-7) + (C-12) penthiopyrad | 2:1 | 500 + 250 | 94 | 88 |
| (A-7) + (D-8) fluoxastrobin | 2:1 | 1000 + 500 | 94 | 78 |
| (A-7) + (C-14) N-[2-(1,3-dimethylbutyl)phenyl] -5- | 2:1 | 500 + 250 | 89 | 67 |
| fluoro-1,3-dimethy1-1H-pyrazole-4-carboxamide | | | | |
| (A-7) + (B-7) difenoconazole | 1:1 | 250 + 250 | 89 | 78 |
| (A-7) + (C-41) isopyrazam | 2:1 | 1000 + 500 | 89 | 78 |

*found = activity found
**calc. = activity calculated according to the Colby formula

### Example Q

### Leptosphaeria nodorum Test (Wheat)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are sprayed with spores from a spore suspension of Leptosphaeria nodorum. The plants remain in an incubation cabin at 20° C. and 100% relative atmospheric humidity for 48 hours. The plants are placed in a greenhouse at a temperature of about 22° C. and a relative atmospheric humidity of about 80%. Evaluation is carried out 8 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE Q
Leptosphaeria nodorum test (wheat)/protective

| | | Active compound | Efficacy in % | |
|---|---|---|---|---|
| Active compound/active compound combination | | application rate in ppm | found* | calc.** |
| (A-7) | | 250 | 0 | |
| (D-13) pyraclostrobin | | 62.5 | 94 | |
| (A-7) + (D-13) pyraclostrobin | 4:1 | 250 + 62.5 | 100 | 94 |

*found = activity found
**calc. = activity calculated according to the Colby formula

### Example R

### Leptosphaeria nodorum Test (Wheat)/Curative

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for curative activity, young plants are sprayed with a spore suspension of Leptosphaeria nodorum. The plants remain in an incubation cabin at 20° C. and 100% relative atmospheric humidity for 48 hours and are then sprayed with the active compound preparation at the stated application rate. The plants are placed in a greenhouse at a temperature of about 22° C. and a relative atmospheric humidity of about 80%. Evaluation is carried out 8 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE R
Leptosphaeria nodorum test (wheat)/curative

| | | Active compound | Efficacy in % | |
|---|---|---|---|---|
| Active compound/active compound combination | | application rate in ppm | found* | calc.** |
| (A-7) | | 250 | 0 | |
| (D-4) azoxystrobin | | 62.5 | 60 | |
| (A-7) + (D-4) azoxystrobin | 4:1 | 250 + 62.5 | 80 | 60 |

*found = activity found
**calc. = activity calculated according to the Colby formula

### Example S

### Fusarium graminearum Test (Barley)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are sprayed with a spore suspension of Fusarium graminearum. The plants are placed in a greenhouse chamber under a translucent incubation hood at 10° C. and 100% relative atmospheric humidity. Evaluation is carried out 5 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE S
Fusarium graminearum test (barley)/protective

| | | Active compound | Efficacy in % | |
|---|---|---|---|---|
| Active compound/active compound combination | | application rate in ppm | found* | calc.** |
| (A-7) | | 1000 | 38 | |
| (B-39) prochloraz | | 500 | 63 | |
| (A-7) + (B-39) prochloraz | 2:1 | 1000 + 500 | 88 | 77 |

*found = activity found
**calc. = activity calculated according to the Colby formula

### Example T

### Puccinia triticina Test (Wheat)/Curative

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for curative activity, young plants are sprayed with a spore suspension of Puccinia triticina. The plants remain in an incubation cabin at 20° C. and 100% relative atmospheric humidity for 48 hours and are then sprayed with the active compound preparation at the stated application rate. The plants are placed in a greenhouse at a temperature of about 20° C. and a relative atmospheric humidity of about 80%. Evaluation is carried out 8 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE T
Puccinia triticina test (wheat)/protective

| | | Active compound | Efficacy in % | |
|---|---|---|---|---|
| Active compound/active compound combination | | application rate in ppm | found* | calc.** |
| (A-7) | | 1000 | 0 | |
| | | 500 | 0 | |
| (D-15) trifloxystrobin | | 500 | 83 | |
| (B-26) imazalil | | 500 | 83 | |
| (B-46) spiroxamine | | 1000 | 40 | |
| (A-7) + (D-15) trifloxystrobin | 1:0.5 | 1000 + 500 | 100 | 83 |
| (A-7) + (B-26) imazalil | 1:1 | 500 + 500 | 100 | 83 |
| (A-7) + (B-46) spiroxamine | 1:1 | 1000 + 1000 | 60 | 40 |

*found = activity found
**calc. = activity calculated according to the Colby formula

### Example U

### Pyrenophora teres Test (Barley)/Curative

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for curative activity, young plants are sprayed with a spore suspension of Pyrenophora teres. The plants remain in an incubation cabin at 20° C. and 100% relative atmospheric humidity for 48 hours and are then sprayed with the active compound preparation of the stated application rate. The plants are placed in a greenhouse at a temperature of about 20° C. and a relative atmospheric humidity of about 80%. Evaluation is carried out 8 days after inoculation. Here, 0% means an efficacy which corresponds to that of the control, whereas an efficacy of 100% means that no infection is observed. The table below clearly shows that the activity found for the active compound combination according to the invention is greater than the calculated activity, i.e. that a synergistic effect is present.

TABLE U
Pyrenophora teres test (barley)/curative

| | | Active compound | Efficacy in % | |
|---|---|---|---|---|
| Active compound/active compound combination | | application rate in ppm | found* | calc.** |
| (A-7) | | 1000 | 11 | |
| | | 1000 | 10 | |
| | | 250 | 11 | |
| (F-4) chlorothalonil | | 1000 | 30 | |
| (B-47) tebuconazole | | 250 | 60 | |
| (G-60) fludioxonil | | 250 | 78 | |
| (B-12) epoxiconazole | | 500 | 78 | |
| (B-41) prothioconazole | | 125 | 78 | |
| (A-7) + (F-4) chlorothalonil | 1:1 | 1000 + 1000 | 60 | 37 |
| (A-7) + (B-47) tebuconazole | 4:1 | 1000 + 250 | 70 | 64 |
| (A-7) + (G-60) fludioxonil | 1:1 | 250 + 250 | 100 | 80 |
| (A-7) + (B-12) epoxiconazole | 2:1 | 1000 + 500 | 94 | 80 |
| (A-7) + (B-41) prothioconazole | 2:1 | 250 + 125 | 100 | 80 |

*found = activity found
**calc. = activity calculated according to the Colby formula

1. A method of controlling microorganisms for crop protection, the method comprising:
applying to a seed, plant, or plant fruit one or more polymeric guanidinium hydroxides based on a diamine which contains oxyalkylene chains and/or alkylene groups inbetween two amino groups, which is obtained by polycondensation of a guanidine acid addition salt with the diamine, giving a polycondensation product in salt form which is then converted by basic anion exchange into the hydroxide form.

2. The method according to claim 1, wherein the polymeric guanidinium hydroxide is poly[2-(2-ethoxyethoxyethyl)guanidinium hydroxide] having at least 3 guanidinium radicals.

3. The method according to claim 1, wherein the polycondensation product in salt form is obtainable by polycondensation of a guanidine acid addition salt with an alkylenediamine and an oxyalkylenediamine in a molar ratio of from 4:1 to 1:4 (alkylenediamine/oxyalkylenediamine).

4. The method according to claim 1, wherein the diamine is an alkylenediamine of the general formula 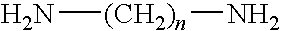
in which n is an integer from 2 to 10.

5. The method according to any of claim 1, wherein the oxyalkylenediamine is a compound of the general formula 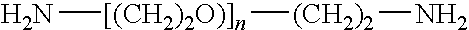
in which m is an integer from 2 to 5.

6. The method according to any of claims 1, wherein the mean molecular mass of the polymeric guanidinium hydroxide is in the range of from 500 to 3000.

7. The method of claim 1, wherein the applied polymeric guanidinium hydroxide is sufficient for controlling phytopathogenic fungi for crop protection.

8. The method according to claim 7, wherein treatment of leaves includes polymeric guanidinium hydroxide from 0.1 to 10 000 g/ha and/or treatment of seed includes polymeric guanidinium hydroxide from 2 to 200 g per 100 kg of seed are employed.

9. An active compound combination comprising:
at least one polymeric guanidinium hydroxide based on a diamine which contains oxyalkylene chains and/or alkylene groups inbetween two amino groups, which is obtained by polycondensation of a guanidine acid addition salt with the diamine, giving a polycondensation product in salt form which is then converted by basic anion exchange into the hydroxide form; and
at least one further fungicidally active compound.

10. The active compound combination according to claim 9, wherein the active compound combination is fungicidally active.

11. The active compound combination according to claim 9, wherein characterized in that the further fungicidally active compound is selected from the group consisting of
(B) inhibitors (or salts thereof) of ergosterol biosynthesis, such as (B-1) aldimorph, (B-2) azaconazole, (B-3) bitertanol, (B-4) bromuconazole, (B-5) cyproconazole, (B-6) diclobutrazole, (B-7) difenoconazole, (B-8) diniconazole, (B-9) diniconazole-M, (B-10) dodemorph, (B-11) dodemorph acetate, (B-12) epoxiconazole, (B-13) etaconazole, (B-14) fenarimol, (B-15) fenbuconazole, (B-16) fenhexamid, (B-17) fenpropidin, (B-18) fenpropimorph, (B-19) fluquinconazole, (B-20) flurprimidol, (B-21) flusilazole, (B-22) flutriafol, (B-23) furconazole, (B-24) furconazole-cis, (B-25) hexaconazole, (B-26) imazalil, (B-27) imazalil sulphate, (B-28) imibenconazole, (B-29) ipconazole, (B-30) metconazole, (B-31) myclobutanil, (B-32) naftifine, (B-33) nuarimol, (B-34) oxpoconazole, (B-35) paclobutrazole, (B-36) pefurazoate, (B-37) penconazole, (B-38) piperalin, (B-39) prochloraz, (B-40) propiconazole, (B-41) prothioconazole, (B-42) pyributicarb, (B-43) pyrifenox, (B-44) quinconazole, (B-45) simeconazole, (B-46) spiroxamine, (B-47) tebuconazole, (B-48) terbinafine, (B-49) tetraconazole, (B-50) triadimefon, (B-51) triadimenol, (B-52) tridemorph, (B-53) triflumizole, (B-54) triforine, (B-55) triticonazole, (B-56) uniconazole, (B-57) viniconazole, (B-58) voriconazole, (B-59) 1-(4-chlorophenyl)-2-(1H-1,2,4-triazol-1-yl)cycloheptanol, (B-60) methyl 1-(2,2-dimethyl-2,3-dihydro-1H-inden-1-yl)-1H-imidazole-5-carboxylate, and (B-61) 0-{1-[(4-methoxyphenoxy)methyl]-2,2-dimethylpropyl}1H-imidazole-1-carbothioate; or
(C) respiratory chain inhibitors (or salts thereof) at complex I or II, such as (C-1) diflumetorim, (C-2) bixafen[N-(3′,4′-dichloro-5-fluoro-1,1′-biphenyl-2-yl)-3-(difluoromethyl)-1-methyl-1H-pyrazole-4-carboxamide], (C-3) boscalid, (C-4) carboxin, (C-5) fenfuram, (C-6) fluopyram, (C-7) flutolanil, (C-8) furametpyr, (C-9) furmecyclox, (C-10) mepronil, (C-11) oxycarboxin, (C-12) penthiopyrad, (C-13) thifluzamide, (C-14) N-[2-(1,3-dimethylbutyl)phenyl]-5-fluoro-1,3-dimethyl-1H-pyrazole-4-carboxamide, (C-15) N-{2-[1,1′-bi(cyclopropyl)-2-yl]phenyl}-3-(difluoromethyl)-1-methyl-1H-pyrazole-4-carboxamide, (C-16) 3-(difluoromethyl)-N-[(9R)-9-isopropyl-1,2,3,4-tetrahydro-1,4-methanonaphthalen-5-yl]-1-methyl-1H-pyrazole-4-carboxamide, (C-17) 3-(difluoromethyl)-N-[(9S)-9-isopropyl-1,2,3,4-tetrahydro-1,4-methanonaphthalen-5-yl]-1-methyl-1H-pyrazole-4-carboxamide, (C-18) 1-methyl-N-[2-(1,1,2,2-tetrafluoroethoxy)phenyl]-3-(trifluoromethyl)-1H-pyrazole-4-carboxamide, (C-19) 3-(difluoromethyl)-1-methyl-N-[2-(1,1,2,2-tetrafluoroethoxy)phenyl]-1H-pyrazole-4-carboxamide, (C-20) 1-methyl-3-(trifluoromethyl)-N-[2′-(trifluoromethyl)biphenyl-2-yl]-1H-pyrazole-4-carboxamide, (C-21) N-(4′-chlorobiphenyl-2-yl)-3-(difluoromethyl)-1-methyl-1H-pyrazole-4-carboxamide, (C-22) N-(2′,4′-dichlorobiphenyl-2-yl)-3-(difluoromethyl)-1-methyl-1H-pyrazole-4-carboxamide, (C-23) 3-(difluoromethyl)-1-methyl-N-[4′-(trifluoromethyl)biphenyl-2-yl]-1H-pyrazole-4-carboxamide, (C-24) N-(2′,5′-difluorobiphenyl-2-yl)-1-methyl-3-(trifluoromethyl)-1H-pyrazole-4-carboxamide, (C-25) 3-(difluoromethyl)-N-[4′-(3,3-dimethylbut-1-yn-1-yl)biphenyl-2-yl]-1-methyl-1H-pyrazole-4-carboxamide, (C-26) 3-(difluoromethyl)-1-methyl-N-(4′-prop-1-yn-1-ylbiphenyl-2-yl)-1H-pyrazole-4-carboxamide, (C-27) 5-fluoro-1,3-dimethyl-N-(4′-prop-1-yn-1-ylbiphenyl-2-yl)-1H-pyrazole-4-carboxamide, (C-28) 2-chloro-N-(4′-prop-1-yn-1-ylbiphenyl-2-yl)nicotinamide, (C-29) N-[4′-(ethynylbiphenyl-2-yl]-3-(difluoromethyl)-1-methyl-1H-pyrazole-4-carboxamide, (C-30) N-(4′-ethynylbiphenyl-2-yl)-5-fluoro-1,3-dimethyl-1H-pyrazole-4-carboxamide, (C-31) 2-chloro-N-(4′-ethynylbiphenyl-2-yl)nicotinamide, (C-32) 3-(difluoromethyl)-N-[4′-(3,3-dimethylbut-1-yn-1-yl)biphenyl-2-yl]-1-methyl-1H-pyrazole-4-carboxamide, (C-33) N-[4′-(3,3-dimethylbut-1-yn-1-yl)biphenyl-2-yl]-5-fluoro-1,3-dimethyl-1H-pyrazole-4-carboxamide, (C-34) 2-chloro-N-[4′-(3,3-dimethylbut-1-yn-1-yl)biphenyl-2-yl]nicotinamide, (C-35) 4-(difluoromethyl)-2-methyl-N-[4′-(trifluoromethyl)-1,1′-biphenyl-2-yl]-1,3-thiazole-5-carboxamide, (C-36) 5-fluoro-N-[4′43-hydroxy-3-methylbut-1-yn-1-yl)biphenyl-2-yl]-1,3-dimethyl-1H-pyrazole-4-carboxamide, (C-37) 2-chloro-N-[4′-(3-hydroxy-3-methylbut-1-yn-1-yl)biphenyl-2-yl]nicotinamide, (C-38) 3-difluoromethyl-N-[4′-(3-methoxy-3-methylbut-1-yn-1-yl)biphenyl-2-yl]-1-methyl-1H-pyrazole-4-carboxamide, (C-39) 5-fluoro-N-[4′-(3-methoxy-3-methylbut-1-yn-1-yl)biphenyl-2-yl]-1,3-dimethyl-1H-pyrazole-4-carboxamide, (C-40) 2-chloro-N-[4′-(3-methoxy-3-methylbut-1-yn-1-yl)biphenyl-2-yl]nicotinamide, and (C-41) isopyrazam; or
(D) respiratory chain inhibitors (or salts thereof) at complex III, such as (D-1) famoxadone, (D-2) fenamidone, (D-3) amisulbrom, (D-4) azoxystrobin, (D-5) cyazofamid, (D-6) dimoxystrobin, (D-7) enestrobin, (D-8) fluoxastrobin, (D-9) kresoxim-methyl, (D-10) metominostrobin, (D-11) orysastrobin, (D-12) picoxystrobin, (D-13) pyraclostrobin, (D-14) pyribencarb, (D-15) trifloxystrobin, (D-16) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (D-17) (2E)-2-(2-{[6-(3-chloro-2-methylphenoxy)-5-fluoro-4-pyrimidinyl]oxy}phenyl)-2-(methoxyimino)-N-methylethanamide, (D-18) 2-chloro-N-(2,3-dihydro-1,1,3-trimethyl-1H-inden-4-yl)-3-pyridinecarboxamide, (D-19) (2E)-2-(methoxyimino)-N-methyl-2-(2-{[({(1E)-1-[3-(trifluoromethyl)phenyl]ethylidene}amino)oxy]methyl}phenyl)ethanamide, (D-20) N-(3-ethyl-3,5,5-trimethylcyclohexyl)-3-(formylamino)-2-hydroxybenzamide, (D-21) (2E)-2-(methoxyimino)-N-methyl-2-{2-[(E)-({1-[3-(trifluoromethyl)phenyl]ethoxy}imino)methyl]phenyl}ethanamide, and (D-22) (2E)-2-[({[(1E)-1-(3-{[(E)-1-fluoro-2-phenylvinyl]oxy}phenyl)ethylidene]aminooxy)methyl]phenyl}-2-(methoxyimino)-N-methylacetamide; or
(E) inhibitors (or salts thereof) of mitosis and cell division, such as (E-1) benomyl, (E-2) carbendazim, (E-3) chlorfenazole, (E-4) diethofencarb, (E-5) ethaboxam, (E-6) fluopicolide, (E-7) fuberidazole, (E-8) pencycuron, (E-9) profenofos, (E-10) thiabendazole, (E-11) thiophanate, (E-12) thiophanate-methyl, (E-13) zoxamide, and (E-14) 5-chloro-6-(2,4,6-trifluorophenyl)-7-(4-methylpiperidin-1-yl) [1,2,4]triazolo[1,5-a]pyrimidine; or
(F) multisite active compounds (or salts thereof), such as (F-1) Bordeaux mixture, (F-2) captafol, (F-3) captan, (F-4) chlorothalonil, (F-5) copper hydroxide, (F-6) copper naphthenate, (F-7) copper oxide, (F-8) copper oxychloride, (F-9) copper sulphate, (F-10) dichlofluanid, (F-11) dithianon (F-12) dodine (F-13) ferbam, (F-14) fluorofolpet, (F-15) folpet, (F-16) guazatine, (F-17) iminoctadine, (F-18) iminoctadine triacetate, (F-19) mancopper, (F-20) mancozeb, (F-21) maneb, (F-22) metiram, (F-23) oxine-copper, (F-24) propineb, (F-25) sulphur and sulphur preparations including calcium polysulphide, (F-26) thiram, (F-27) tolylfluanid, (F-28) zineb, and (F-29) ziram; or
(G) the other fungicides (or salts thereof) below, such as (G-1) acibenzolar-5-methyl, (G-2) isotianil, (G-3) probenazole, (G-4) tiadinil, (G-5) andoprim, (G-6) blasticidin-S, (G-7) cyprodinyl, (G-8) kasugamycin, (G-9) mepanipyrim, (G-10) pyrimethanil, (G-11) fentin acetate, (G-12) fentin chloride, (G-13) fentin hydroxide, (G-14) silthiofam, (G-15) benthiavalicarb, (G-16) dimethomorph, (G-17) flumorph, (G-18) iprovalicarb, (G-19) mandipropamid, (G-20) valiphenal, (G-21) polyoxins, (G-22) polyoxorim, (G-23) validamycin A, (G-24) biphenyl, (G-25) chloroneb, (G-26) chlozolinate, (G-27) edifenfos (G-28) etridiazole, (G-29) iodocarb, (G-30) iprobenfos, (G-31) iprodione, (G-32) isoprothiolane, (G-33) procymidone, (G-34) propamocarb, (G-35) propamocarb hydrochloride, (G-36) prothiocarb, (G-37) pyrazophos, (G-38) tolcofos-methyl, (G-39) vinclozolin, (G-40) carpropamid, (G-41) diclocymet, (G-42) fenoxanil, (G-43) phthalide, (G-44) pyroquilon, (G-45) tricyclazole, (G-46) benalaxyl, (G-47) benalaxyl-M, (G-48) bupirimate, (G-49) clozylacon, (G-50) dimethirimol, (G-51) ethirimol, (G-52) furalaxyl, (G-53) hymexazole, (G-54) metalaxyl, (G-55) metalaxyl-M (Mefenoxam), (G-56) ofurace, (G-57) oxadixyl, (G-58) oxolinic acid, (G-59) fenpiclonil, (G-60) fludioxonil, (G-61) quinoxyfen, (G-62) binapacryl, (G-63) dinocap, (G-64) fluazinam, (G-65) meptyldinocap, (G-66) benthiazole, (G-67) bethoxazin, (G-68) capsimycin, (G-69) carvone, (G-70) chinomethionat, (G-71) cufraneb, (G-72) cyflufenamid, (G-73) cymoxanil, (G-74) cyprosulfamide, (G-75) dazomet, (G-76) debacarb, (G-77) dichlorophen, (G-78) diclomezine, (G-79) dicloran, (G-80) difenzoquat, (G-81) diphenylamine, (G-82) ferimzone, (G-83) flumetover, (G-84) fluoroimide, (G-85) flusulfamide, (G-86) fosetyl-Al, (G-87) fosetyl-calcium, (G-88) fosetyl-sodium, (G-89) hexachlorobenzene, (G-90) irumamycin, (G-91) methasulfocarb, (G-92) methyl isothiocyanate, (G-93) metrafenone, (G-94) mildiomycin, (G-95) natamycin, (G-96) nickel dimethyldithiocarbamate, (G-97) nitrothal-isopropyl, (G-98) octhilinone, (G-99) oxyfenthiin, (G-100) pentachlorophenol, (G-101) phosphorous acid, (G-102) propamocarbfosetyl, (G-103) propanosin-sodium, (G-104) proquinazid, (G-105) pyrrolnitrins, (G-106) quintozene, (G-107) tecloftalam, (G-108) tecnazene, (G-109) triazoxide, (G-110) trichlamide, (G-111) zarilamid, (G-112) 8-hydroxyquinoline sulphate, (G-113) 2,3,5,6-tetrachloro-4-(methylsulphonyl)pyridine, (G-114) 3,4,5-trichloropyridine-2,6-dicarbonitrile, (G-115) 3-[5-(4-chlorophenyl)-2,3-dimethylisoxazolidin-3-yl]pyridine, (G-116) N-(4-chloro-2-nitrophenyl)-N-ethyl-4-methylbenzenesulphonamide, (G-117) 2,3-dibutyl-6-chlorothieno[2,3-d]pyrimidin-4(3H)one, (G-118) 2-butoxy-6-iodo-3-propylbenzopyran-4-one, (G-119) N-(4-chlorobenzyl)-3-[3-methoxy-4-(prop-2-yn-1-yloxy)phenyl]propanamide, (G-120) N-(6-methoxy-3-pyridinyl)cyclopropanecarboxamide, (G-121) N-[(4-chlorophenyl)(cyano)methyl]-3-[3-methoxy-4-(prop-2-yn-1-yloxy)phenyl]propanamide, (G-122) N-[(5-bromo-3-chloropyridin-2-yl)methyl]-2,4-dichloropyridine-3-carboxamide, (G-123) N-[1-(5-bromo-3-chloropyridin-2-yl)ethyl]-2,4-dichloropyridine-3-carboxamide, (G-124) N-[1-(5-bromo-3-chloropyridin-2-yl)ethyl]-2-fluoro-4-iodopyridine-3-carboxamide, (G-125) N-[4-(3-tert-butyl-4-chlorophenoxy)-2,5-dimethylphenyl]-N-ethyl-N-methylimidoformamide, (G-126) N-[4-(3-tert-butyl-4-chlorophenoxy)-2-methyl-5-(trifluoromethyl)phenyl]-N-ethyl-N-methylimidoformamide, (G-127) N-[4-(3-tert-butyl-4-chlorophenoxy)-5-(difluoromethyl)-2-methylphenyl]-N-ethyl-N-methylimidoformamide, (G-128) N′-[4-(3-tert-butyl-4-fluorophenoxy)-2,5-dimethylphenyl]-N-ethyl-N-ethylimidoformamide, (G-129) N′-[4-(3-tert-butyl-4-fluorophenoxy)-2-methyl-5-(trifluoromethyl)phenyl]-N-ethyl-N-methylimidoformamide, (G-130) N-[4-(3-tert-butyl-4-fluorophenoxy)-5-(difluoromethyl)-2-methylphenyl]-N-ethyl-N-methylimidoformamide, (G-131) N′-[4-(4-chloro-3-isopropylphenoxy)-2,5-dimethylphenyl]-N-ethyl-N-methylimidoformamide, (G-132) N′-[4-(4-chloro-3-isopropylphenoxy)-2-methyl-5-(trifluoromethyl)phenyl]-N-ethyl-N-methylimidoformamide, (G-133) N-[4-(4-chloro-3-isopropylphenoxy)-5-(difluoromethyl)-2-methylphenyl]-N-ethyl-N-methylimidoformamide, (G-134) N′-{4-[(3-tert-butyl-1,2,4-thiadiazol-5-yl)oxy]-2,5-dimethylphenyl}-N-ethyl-N-methylimidoformamide, (G-135) N′-{4-[(3-tert-butyl-1,2,4-thiadiazol-5-yl)oxy]-2-methyl-5-(trifluoromethyl)phenyl}-N-ethyl-N-methylimidoformamide, (G-136) N-{4-[(4-tert-butyl-1,3-thiazol-2-yl)oxy]-2,5-dimethylphenyl}-N-ethyl-N-methylimidoformamide, (G-137) N′-{4-[(4-tert-butyl-1,3-thiazol-2-yl)oxy]-2-methyl-5-(trifluoromethyl)phenyl}-N-ethyl-N-methylimidoformamide, (G-138) N′-{5-(difluoromethyl)-2-methyl-4-[3-(trimethylsilyl)propoxy]phenyl}-N-ethyl-N-methylimidoformamide, (G-139) N-ethyl-N′-[4-(4-fluoro-3-isopropylphenoxy)-2,5-dimethylphenyl]-N-ethylimidoformamide, (G-140) N-ethyl-N′-[4-(4-fluoro-3-isopropylphenoxy)-2-methyl-5-(trifluoromethyl)phenyl]-N-methylimidoformamide, (G-141) N-ethyl-N′-[4-(4-fluoro-3-isopropylphenoxy)-2-methyl-5-(trifluoromethyl)phenyl]-N-methylimidoformamide, (G-142) N-ethyl-N′-{4-[(3-isopropyl-1,2,4-thiadiazol-5-yl)oxy]-2,5-dimethylphenyl}-N-methylimidoformamide, (G-143) N-ethyl-N′-{4-[(3-isopropyl-1,2,4-thiadiazol-5-yl)oxy]-2-methyl-5-(trifluoromethyl)phenyl}-N-methylimidoformamide, (G-144) N-ethyl-N′-{4-[4-isopropyl-1,3-thiazol-2-yl)oxy]-2,5-dimethylphenyl}-N-methylimidoformamide, (G-145) N-ethyl-N′-{4-[(4-isopropyl-1,3-thiazol-2-yl)oxy]-2-methyl-5-(trifluoromethyl)phenyl}-N-methylimidoformamide, (G-146) N-ethyl-N-methyl-N′-{2-methyl-5-(trifluoromethyl)-4-[3-(trimethylsilyl)propoxy]-phenyl}imidoformamide, and (G-147) S-allyl 5-amino-2-isopropyl-4-(2-methylphenyl)-3-oxo-2,3-dihydro-1H-pyrazole-1-carbothioate.

12. A composition comprising active compound combinations according to claim 9.

13. A composition according to claim 12 comprising further auxiliaries, solvents, carriers, surfactants or extenders.

14. A method for controlling phytopathogenic fungi for crop protection, the method comprising:
applying the active compound combination according to claim 9 or composition thereof to the seed, the plant, to fruits of plants or to the soil on which the plant grows or is supposed to grow.

15. The method according to claim 14, wherein the plant, the fruits of plants or the soil on which the plant grows or is intended to grow are treated so as to control phytopathogenic fungi.

16. The method according to claim 14, wherein treatment of leaves includes polymeric guanidinium hydroxide from 0.1 to 10 000 g/ha and/or treatment of seed includes polymeric guanidinium hydroxide from 2 to 200 g per 100 kg of seed are employed.

17. A method for controlling phytopathogenic fungi for crop protection, the method comprising:
applying a composition according to claim 12 to the seed, the plant, to fruits of plants or to the soil on which the plant grows or is supposed to grow.

18. A method for controlling a microorganism, the method comprising:
applying the active compound combination according to claim 9 to a seed, seed of a transgenic plant and/or to a transgenic plant.

19. A seed treated with an active compound combination according to claim 9.

20. A seed treated with polymeric guanidinium hydroxides according to claim 1.
